(19) United States
(12) Patent Application Publication
(10) Pub. No.: US 2017/0328896 A1
(43) Pub. Date: Nov. 16, 2017

(54) ASSAY SYSTEMS FOR POINT OF CARE DETECTION OF OCULAR ANALYTES

(71) Applicant: INSIGHT INSTRUMENTS, INC., Stuart, FL (US)

(72) Inventors: Klaus Peter LULOH, Stuart, FL (US); Ross MCDONOUGH, Stuart, FL (US); Michael ANNEN, Ft. Pierce, FL (US); Jade GOLD, Stuart, FL (US)

(21) Appl. No.: 15/437,346

(22) Filed: Feb. 20, 2017

Related U.S. Application Data
(60) Provisional application No. 62/297,559, filed on Feb. 19, 2016.
(60) Provisional application No. 62/393,993, filed on Sep. 13, 2016.

(51) Int. Cl. G01N 33/558; A61B 10/02; G01N 33/543; G01N 33/68; B01L 3/00; G01N 33/53

(52) U.S. Cl. CPC G01N 33/558; B01L 3/5023; G01N 33/5308; G01N 33/54386; G01N 33/6863; A61B 10/0283; B01L 2300/069; B01L 2300/0672; B01L 2300/0654; B01L 2200/026; B01L 2300/0832; B01L 2300/087; B01L 2400/0406; B01L 2400/0633; G01N 2800/164; G01N 2333/52

(57) ABSTRACT

Disclosed herein are systems and methods for detecting ocular analytes in vitreous humor or aqueous humor. Specifically exemplified are systems having a sample acquisition device that is inline with an analyte detection device. The system embodiments allow for the easy procurement and testing of samples. In a typical embodiment, the analyte detection device includes a sample staging chamber and a test chamber that comprises reagents that specifically interact with the analyte. The test chamber may include a sample pad, a conjugate pad having at least one conjugate reagent specific to the analyte loaded thereon, an assay platform having a substrate with at least one test region having a test reagent immobilized thereon, the test reagent being specific to the analyte; and an optional absorbent pad.

### BACKGROUND

### 1. Field of the Invention

The invention disclosed herein relates to assays, systems and methods for detecting biomarkers for certain ophthalmic diseases, preferably vitreoretinal diseases such as age-related macular degeneration (AMD), central retinal vein occlusion (CRVO) and diabetic retinopathy (PDR). These assays, systems and methods are useful for rapid detection of the biomarkers at the point of care.

### 2. Description of the Background

Vitreoretinal diseases, such as AMD, CRVO, and PDR, are leading causes of blindness and vision loss worldwide. The retina is an extension of the brain that lines the eye interior. It contains the nerve endings (photoreceptors) that allow vision. The vitreous humor (also referred to as the vitreous or the vitreous body) is a clear, gelatinous substance that fills the eye behind the lens, contacts the retina, and helps to keep the retina in place. In vitreoretinal disease states, markers such as VEGF (vascular endothelial growth factor), IL-6 (interleukin 6), or MCP-1 (monocyte chemotactic protein 1) can be detected in the vitreous. The presence and amount of such markers in the vitreous can be used to diagnose and to determine the stage or severity of these conditions, including AMD, RVO, PDR, and trauma.

Vascular endothelial growth factor (VEGF) is a signaling molecule that stimulates angiogenesis and can be detected in a number of disease states, including cancer and vitreoretinal diseases like AMD and PDR. IL-6 is a pro-inflammatory cytokine that stimulates an immune response after trauma, infection, cancer, and the like, and also can be detected in vitreoretinal disease. MCP-1 (also sometimes known as small inducible cytokine A2 or chemokine (C-C motif) ligand 2) is a cytokine in the CC chemokine family that recruits immune cells to the sites of inflammation and is produced by tissue injury or infection, for example of tissues in the central nervous system, and also can be detected in vitreoretinal diseases.

Antibody drugs such as Avastin™, Lucentis™, and Eyelea™ show promising results in the treatment of those diseases. However, the treatment regimens with these anti-VEGF or anti cytokine drugs consist of intravitreal injections at fixed (frequently monthly) intervals for sometimes unlimited duration at great financial cost to the patient and adverse effects on the patient's quality of life. Being able to measure and monitor the levels of biomarkers such as VEGF, therefore can greatly benefit patients suspected of suffering from vitreoretinal disease or being treated for vitreoretinal disease because it allows more accurate diagnosis of the disease state and better determination of the effectiveness or potential effectiveness of treatment.

Currently, determination of biomarker levels is performed using analytic biochemistry assays, such as ELISA (enzyme-linked immunosorbent assay) or modified version thereof. Such assays are described in United States Patent Application serial no. 2014/0193840 A1. These types of assays are complicated to perform, require trained personnel, are expensive, and require time to perform. Therefore, treatment generally must be begun before the results of the test are obtained, preventing their use for diagnostic purposes. There is a need in the art for rapid testing of ocular biomarkers which can be performed at the point of care with less cost, so that the tests can be used for diagnostic purposes and completed prior to treatment. Such a simple and rapid testing method would reduce unnecessary treatment or re-treatment, thereby also reducing treatment cost and potential complications of treatment.

### SUMMARY

The present application describes and claims an assay, method and system for detecting biomarker cytokine levels in a biological sample, preferably a sample obtained from the eye. The sample can pertain to vitreous humor, aqueous humor, eye tissue or a combination thereof, and preferably is a sample of vitreous humor.

Embodiments of the invention provided herein enable quick detection and monitoring of biomarker levels in a biological sample. These tests can determine whether treatment should be given or to determine the efficacy of the treatment. With this information, unnecessary treatment rates and unnecessary repeat intravitreal injections can be avoided, reducing costs and complications (for example due to injection as well as systemic toxicity), and increasing quality of life for the patient. A specific embodiment of a system includes a portable vitrectomy system with a sharp-tipped disposable probe, in-line sample collection device with integrated LFIA (lateral flow immunoassay) test, and hand-held reader.

A general embodiment disclosed herein pertains to a system for detecting an analyte in a vitreous humor sample, that includes a vitreous humor acquisition device having a cannulated needle with an aspiration inlet; an aspiration conduit in fluid communication with the aspiration inlet; an aspirator for applying a vacuum to the aspiration conduit; and an analyte detection device in fluid communication with the aspirator and the acquisition device. The cannulated needle includes an elongated body and tapered distal tip, wherein the aspiration inlet is positioned on the elongated body upstream from the tapered distal tip.

The analyte detection device may include a sample staging chamber and a test chamber that comprises reagents that specifically interact with the analyte. The sample staging chamber is typically in fluid communication with the aspirator and the aspiration conduit, and the analyte detection device may further include a valve positioned between the sample staging chamber and the test chamber. The valve may be a manual valve or a one-way valve such that is closed during aspiration of the vitreous humor sample. The test chamber may include a sample pad, a conjugate pad having at least one conjugate reagent specific to the analyte loaded thereon, an assay platform comprising a substrate with at least one test region having a test reagent immobilized thereon with the test reagent being specific to the analyte. The analyte detection device may further comprise an absorbent pad. The conjugate reagent may be labeled as is discussed further herein. The system embodiment may be configured such that the vitreous sample contacts the sample pad to allow fluid in the vitreous sample to flow from the sample pad to the conjugate pad and to the assay platform by capillary action. The conjugate reagent and test reagent may take the form of antibodies or aptamers that bind to different epitopes or a common epitope of the analyte. The assay platform comprises a control region having a control binding reagent immobilized thereon, wherein the control binding reagent specifically binds to the at least one conjugate reagent. In a more specific version, the assay platform includes a first test region with a first test reagent specific to a first analyte and a second test region with a second test reagent specific to a second analyte.

In a specific embodiment, disclosed is a system for detecting an analyte in a vitreous humor sample, the system including a vitreous humor acquisition device including a cannulated needle with an aspiration inlet and an aspiration tube in fluid communication with the aspiration inlet. The aspiration conduit has a body with distal end engaged to the acquisition device and a proximal end comprising a first connector. The embodiment also has a sample tube comprising a body with a distal end and a proximal end, wherein the distal end has a second connector that connects to the first connector. The system may have an aspirator engaged to the proximal end of the sampling tube, wherein the aspirator implements a valve for shutting off aspiration. The sampling tube may include at least one indicator positioned at a predetermined location on the body of the sampling tube. In a more specific version, the at least one indicator involves two indicators that are each positioned on the body of the sampling tube to provide a visual feedback of a desired sample volume. The proximal end of the sampling tube may engage to the valve.

The embodiments described in the preceding paragraph may further include an analyte detection device that has a housing with a proximal end and distal end. The proximal end has a port, wherein upon disconnecting the first and second connectors, the second connector connects to the port of the analyte detection device. In a more specific version, the analyte detection device has a sample staging chamber in fluid communication with the port and an overflow passage in fluid communication with the sample staging chamber. The analyte detection device may also include a sample transfer chamber, a test chamber and a sample pad that is disposed in the sample staging chamber, sample transfer chamber and test chamber for transferring a sample from the sample staging chamber to the test chamber. The device may also include a conjugate pad disposed in the sample transfer chamber and test chamber that contacts the sample pad. In another version, an assay platform is disposed in the test chamber that contacts the conjugate pad. The assay platform may have a test region and a control region. The analyte detection device may also include a wicking chamber and a wicking pad disposed within the wicking chamber and test chamber, wherein the wicking pad contacts the assay platform. The analyte detection device may also comprise a vent in fluid communication with the overflow passage.

Also provided is a method for detecting at least one analyte in a vitreous sample using a system embodiment as described above. The method involves aspirating the vitreous sample by applying vacuum to the sampling tube while the first and second connectors are connected and the sample acquisition device is in an eye. When the vitreous sample reaches the at least one indicator, the valve is closed and the first and second connectors are disconnected and the second connector is engaged to the port. Pressured is then applied to the sampling tube to deliver the vitreous sample to the analyte detection device.

### BRIEF DESCRIPTION OF THE DRAWINGS

Embodiments of the present invention are illustrated by way of example, and not by way of limitation, in the figures of the accompanying drawings below.

FIG. 1 shows a side perspective view of point of care device for detection/evaluating analytes in a sample.

FIG. 2 shows a partial cross-section view of the embodiment shown in FIG. 1 along axis C-C.

FIG. 3 shows a partial cross-section view of an alternative component that can be implemented in the point of care device in FIG. 1.

FIG. 4A shows a partial side cross-section view of the embodiment of FIG. 4B.

FIG. 4B shows a top cross-section view of a point of care device embodiment that includes a sample chamber and test chamber.

FIG. 5A shows a partial side cross-section view of a point of care device embodiment having two valves for controlling flow of sample.

FIG. 5B shows section of the view in FIG. 5A with valves turned to encourage sample flow into the test chamber.

FIG. 5C shows a perspective view of a valve component embodiment.

FIG. 6A shows a side perspective view of a point of care device for detecting/evaluating an analyte in a sample.

FIG. 6B shows a view of the embodiment in FIG. 6A with the conduit portions missing to show the movement of inner sample chamber.

FIG. 7 shows a prior art LFIA device.

FIG. 8 shows a sample acquisition and analyte detection system embodiment.

FIG. 9 is a diagram depicting a sample acquisition and analyte detection system embodiment.

FIG. 10 is a diagram depicting a sample acquisition and analyte detection system embodiment.

FIG. 11 shows a side perspective view of a further sample acquisition and manipulation system embodiment.

FIG. 12. is a cross-sectional view of an analyte detection device that is configured to interact with a sample acquisition device.

FIG. 13 Is a graph showing the relationship between displaced syringe volume and pressure.

### DETAILED DESCRIPTION OF THE INVENTION

### 1. Introduction

The invention is described herein with reference to specific embodiments thereof. Various modifications and changes, however, can be made to the invention without departing from the broader spirit and scope of the invention. The specification and drawings are, accordingly, illustrative rather than restrictive. Throughout this specification and the claims, unless the context requires otherwise, the word “comprise” and its cognates, such as “comprises” and “comprising,” imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Unless otherwise clear from the context, a numerical value presented herein has an implied precision given by the least significant digit. Thus a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Embodiments are described herein with reference to the attached figures wherein like reference numerals are used throughout the figures to designate similar or equivalent elements. The figures are not drawn to scale and they are provided merely to illustrate aspects disclosed herein. Several disclosed aspects are described below with reference to non-limiting example applications for illustration. Numerous specific details, relationships, and methods are set forth to provide a full understanding of the embodiments disclosed herein. One having ordinary skill in the relevant art, however, will readily recognize that the disclosed embodiments can be practiced without one or more of the specific details or with other methods. In other instances, well-known structures or operations are not shown in detail to avoid obscuring aspects disclosed herein. The embodiments are not limited by the illustrated ordering of acts or events, as some acts may occur in different orders and/or concurrently with other acts or events. Furthermore, not all illustrated acts or events are required to implement a methodology in accordance with the embodiments.

### 2. Definitions

The following terms as used herein have the following definitions. Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, because measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. The terms front, back and side are only used as a frame of reference for describing components herein and are not to be limiting in any way.

The terms “first,” “second,” and the like, as used herein, do not denote any order, quantity, or importance, but rather are used to distinguish one element from another. The terms “a” and “an” do not denote a limitation of quantity, but rather denote the presence of at least one of the referenced item. The modifier “about” used in connection with a quantity is inclusive of the stated value and has the meaning dictated by the context. All ranges disclosed within this specification are inclusive and are independently combinable. Furthermore, to the extent that the terms “including,” “includes,” “having,” “has,” “with,” or variants thereof are used in either the detailed description and/or the claims, such terms are intended to be inclusive in a manner similar to the term “comprising.”

The term “analyte” refers to any compound or composition to be measured in an assay, for example a biomarker or a portion thereof. Such an analyte also is referred to as a target or target analyte, and is capable of binding specifically to a capture or test reagent, which can be an antigen, hapten, protein, drug, metabolite, nucleic acid, ligand, receptor, enzyme, aptamer (RNA or DNA based, and containing natural or unnatural bases or any combination thereof), antibody (either monoclonal or polyclonal) or fragment thereof, affibody, scFv, sdAb, affimer, avimer, aptide, cell, or any of the aforementioned which are derivatized with a detection molecule (e.g. fluorophore, nanoparticle, quantum dot etc.). Preferably, the analyte is VEGF or a portion thereof, such as an epitope or hapten thereof, or is IL-6 or MCP-1, however any biomarker can be tested and detected using these methods. Analytes also can include antibodies and receptors, including active fragments or fragments thereof. An analyte can include an analyte analogue, which is a derivative of an analyte, such as, for example, an analyte altered by chemical or biological methods, such as by the action of reactive chemicals, such as adulterants or enzymatic activity.

The term “ocular analyte” refers to an analyte directly or indirectly pertaining to a marker related to an eye disease or condition. An ocular analyte may include, but is not limited to, an angiogenic ocular analyte or an inflammatory ocular analyte. Specific examples of angiogenic ocular analytes include, but are not limited to, C-kit Y703, c-kit Y719, MMP-2, MMP-9, retinol binding protein-4 (RBP4), Secreted Protein Acidic and Rich in Cysteine (SPARC), Akt., VEGFR, EGFR, Bcr-Abl, Her2-Neu (erbB2), TGFR, PDGR, PDGFR, FGF, FGF-R, and PEDF. Specific examples of inflammatory ocular analytes include, but are not limited to, BAD Ser112, Bcl-2, C-abl, CC9 D330, Fadd 5194, TNF-alpha, IL-1, IL-1B, IL-6, IL-6R, IL-8, IL-10 and MCP-1. “Indirectly pertaining to a marker” refers to and includes scenarios where the analyte detected is known to correlate with a level of another marker or molecule of interest either in the sample or in the biological context. For example, high levels of mRNA encoding IL-6 in a certain tissue or fluid may be understood to correlate with a known level of IL-6 in the same tissue or fluid, thus detection of this mRNA analyte could provide useful information concerning the level of IL-6. In addition, a marker byproduct can be detected as the analyte as opposed to the marker molecule of interest.

The term “antibody” is used here in its broadest sense refers to an immunoglobulin, or derivative or fragment or active fragment thereof, having an area on the surface or in a cavity which specifically binds to and is thereby defined as complementary with a particular spatial and polar organization of another molecule. The antibody can be monoclonal or polyclonal and can be prepared by techniques that are well known in the art such as, for example, immunization of a host and collection of sera or hybrid cell line technology, or recombinant technology. The term includes monoclonal antibodies, polyclonal antibodies, anti-idiotypic antibodies, synthetic antibodies, multispecific antibodies (e.g., bispecific antibodies) formed from at least two intact antibodies, and antibody fragments so long as they exhibit the desired binding activity. The antibodies can be chimeric antibodies, including humanized antibodies as described in Jones et al., Nature 321:522-525, 1986, Riechmann et al., Nature 332:323-329, 1988, Presta, Curr. Opin. Struct. Biol. 2:593-596, 1992, Vaswani and Hamilton, Ann. Allergy, Asthma & Immunol. 1:105-115, 1998, Harris, Biochem. Soc. Transactions 23:1035-1038, 1995, and Hurle and Gross, Curr. Opin. Biotech. 5:428-433, 1994. Antibodies of any class or isotype (e.g., IgA, IgA1, IgA2, IgD, IgE, IgG, IgG1, IgG2, IgG3, IgG4, and IgM) can be used.

The term “antibody” also refers to any antibody fragment(s) that retain a functional antigen binding region. Examples of antibody fragments include Fab, Fab′, F(ab′)2, and Fv fragments; diabodies; linear antibodies; single-chain antibody molecules; and multispecific antibodies formed from antibody fragments, all of which are known in the art. Papain digestion of antibodies produces two identical antigen-binding fragments, called “Fab” fragments, each with a single antigen-binding site. Pepsin treatment yields an F(ab′)2 fragment that has two antigen-combining sites and is still capable of cross-linking antigen. Fab′ fragments differ from Fab fragments by the addition of a few residues at the carboxy terminus of the heavy chain CH1 domain including one or more cysteines from the antibody hinge region. F(ab′)2 antibody fragments are pairs of Fab′ fragments which have hinge cysteines between them. Other chemical couplings of antibody fragments are also known. “Fv” is the minimum antibody fragment which contains a complete antigen-binding site. Diabodies are described more fully in, for example, European Patent No. 404,097, International Patent Application WO 1993/01161, Hudson et al., Nat. Med. 9:129-134, 2003, and Hollinger et al., PNAS USA 90: 6444-6448, 1993. Triabodies and tetrabodies also are described in Hudson et al., Nat. Med. 9:129-134, 2003.

The term “antibody,” as used herein, also includes antibody substitutes or any natural, recombinant or synthetic molecule that specifically binds with high affinity to a particular target. Thus, the term “antibody” includes such synthetic antibodies or antibody substitutes such as aptamers, affibodies, affimers, avimers, aptides, and the like. Therefore, when describing the assay systems, devices and methods according to embodiments of the invention here, use of the term “antibody” for use as, for example, a reagent in the assay, indicates any of these alternatives also can be used.

The term “aptamer” refers to a nucleic acid or peptide molecule that specifically binds to a molecule of interest (target) with high affinity. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer may be prepared by any known method, including synthetic, recombinant, and purification methods, and may be used alone or in combination with other aptamers specific for the same target.

The term “biomarker” or “ocular biomarker” refers to a detectable biomolecule that appears or increases in amount as an indicator of or marker of a particular disease or condition. In particular, this term includes any naturally occurring and detectable biological molecule, the presence of which, or the presence of which at or above a certain concentration or amount, indicates a vitreoretinal disease. For example, VEGF-A is a signaling molecule that stimulates angiogenesis, which is present in a number of disease states, including vitreoretinal disease (e.g., AMD. PDR) and cancer. Other potential biomarkers contemplated as useful with the present invention include IL-6, MCP-1, IP10, and other pro-angiogenic and/or pro-inflammatory cytokines, and the like, and include the analytes discussed above as angiogenic ocular analytes or inflammatory ocular analytes. The term “biomarker” also can refer to any analyte used to determine the presence or degree of a vitreoretinal disease or condition.

The term “fluid communication” as used herein with respect to one or more components of a system means that fluid (gas, liquid, and/or semi-liquid) can be transferred directly or indirectly from one component to another. Direct fluid communication pertains to fluid transfer from a first component that is contiguously associated with a second component. Indirect fluid communication pertains to fluid transfer from one component to another that involves passage through an intervening component.

The term “immobilized” (with respect to capture reagents or other reagents described herein) means that the migration of the capture molecule on the membrane or other surface on which it is immobilized (e.g., due to capillary flow of fluid such as the sample) or its escape from its immobilized location on the membrane is substantially impeded and, in certain embodiments, completely impeded. Methods for immobilizing the capture reagent are known in the art.

The term “light” as used herein is intended to include a device that can generate of electromagnetic radiation.

The term “operable contact” refers to direct or indirect contact (intervening component) between a first and second component of a test device whereby the contact allows fluid to flow from one component to another via gravity, capillary action or any other fluid flow.

The term “sample” refers to any acquired material to be tested for the presence or amount of an analyte. Preferably, a sample is a fluid sample, preferably a liquid or gel sample. Examples of liquid samples that may be tested using a test device of the present invention include bodily fluids including blood, embryonic fluid, serum, plasma, saliva, urine, ocular fluid, vitreous humor of the eye, aqueous humor of the eye, semen, and spinal fluid; preferably the sample is obtained from the eye. The term “sample” also includes material that has been collected from a subject and treated further, for example solubilized or diluted in a solvent suitable for testing.

The term “reagent” refers to a molecule, typically a binding molecule which is used to detect a target analyte, including reagents that bind to the target, agents that bind to the target-binding reagent, detectably labeled reagents and the like as known for use in immune-type assays. The reagent can be an antibody, an aptamer, an aptide, an avimer, an affibody, an affimer, or any specific binding partner known in the art that can bind the target with high affinity and specificity. A test reagent is an antibody or antibody substitute or other molecular surrogate that specifically binds to the target to be detected, which is immobilized. The test reagent also is referred to as a capture antibody or capture molecule. A conjugate reagent is a specific-binding molecule such as an antibody or aptamer that is conjugated to a detectable label and binds the target at a different site than the test reagent. A control binding reagent is a molecule, such as an antibody or aptamer, that specifically binds to the conjugate reagent.

The term “therapeutic agent” as used herein is a pharmacologic agent useful in treating a disease or condition, preferably an eye disease or condition. These can include both naturally occurring substances (in purified form, partially purified form, or in unpurified form). Naturally occurring substances includes proteins, nucleic acids, fatty acids, steroids and other organic compounds produced in plants, animals, microorganisms or from non-living sources. In another aspect, a therapeutic agent may be a non-naturally occurring substance including proteins, nucleic acids, fatty acids, steroids and other organic compounds. A non-naturally occurring therapeutic agent can include modified natural products or substances without naturally occurring homologues.

Examples of therapeutic agents include antibodies, receptor agonists and antagonists, signaling pathway agonists and antagonists, small molecules, proteins, nucleic acids and other active agents known in the art. A therapeutic agent can be from any class of drugs, such as anti-angiogenesis agents, cancer treatments, anti-inflammation, molecules involved in growth, anti-apoptosis agents, steroid compounds used for reduced swelling, and monoclonal antibodies and fragments thereof. In one aspect, inhibitors of TNFalpha or inhibitors molecules in the signaling pathways of TNF-R1 or TNF-R2 are therapeutic agents.

In another aspect, a therapeutic agent is an inhibitor of VEGF, such as bevacizumab (Avastin®), pegaptanib (Macugen®), ranibizumab (Lucentis®), aflibercept (Eyelea®), Dexamethasone (Decadron®) and triamcinolone (Aristocort®). Bevacizumab is a recombinant humanized mouse monoclonal antibody that binds to and inhibits vascular endothelial growth factor A (VEGF-A). Ranibizumab is a monoclonal antibody fragment (Fab) from the same monoclonal antibody as Avastin, and also binds to VEGF. Aflibercept is a recombinant fusion protein comprising portions of the extracellular domains of human VEGF receptors 1 and 2 fused to the Fc portion of human IgG1, and acts as a soluble decoy receptor that binds VEGF. All of these compositions inhibit the binding and activation of cognate VEGF receptors.

The present invention also provides for identifying a subject who would benefit from administration of a different therapeutic agent. In such an aspect, the therapeutic agent may be something other than the previously administered treatment, for example without limitation, not bevacizumab, not pegaptanib, not ranibizumab, not Dexamethasone, or not triamcinolone.

The term “vitreoretinal disease,” as used herein, refers to any disease of the retina and/or vitreous body of the eye. Such diseases include such conditions as age-related or idiopathic macular degeneration, retinal detachments or tears, retinopathy of prematurity, retinoblastoma, uveitis, cancer of the eye, retinitis pigmentosa, macular holes, CRVO, branch retinal vein occlusion (BRVO), flashes and floaters, proliferative diabetic retinopathy, diffuse retinal thickening, cystoid macular edema, diabetes mellitus, uveal melanoma, proliferative vitreoretinopathy, Eales disease, idiopathic macular hole, and diabetic retinopathy.

### 3. Overview

Embodiments of the invention relate to the quick and accurate determination of certain biomarkers in fluid, gel, or tissue samples from a subject, preferably samples taken from or associated with the eye. Rapid clinical monitoring of important biomarkers of disease, for example inflammatory or pro-angiogenic factors in patients with vitreoretinal disease, is useful to prevent unnecessary repeat treatments (such as intravitreal injections) for patients and to reduce cost for both patients and health care providers.

### 4. Detailed Description of the Invention

A. General Description of System

In general, an analyte detection device for determining the presence or amount of an analyte in a sample includes a sample staging chamber and a test chamber. The test chamber usually includes components and reagents that specifically interact with the analyte, for example a capture molecule that can specifically bind the target analyte to be detected or quantitated. In a specific embodiment, the sample staging chamber is in fluid communication with an aspirator and an aspiration conduit that delivers sample to the analyte detection device. The analyte detection device may also include a valve positioned between the sample staging chamber and the test chamber to facilitate creation of a vacuum during delivery of the sample to the sample chamber, and which subsequently allows the transfer of sample from sample chamber to the test chamber. Typically, the valve is a manual valve or one-way valve that is closed during aspiration.

The test chamber preferably includes (1) a sample pad, (2) a conjugate pad comprising at least one conjugate reagent specific to the analyte loaded onto the conjugate pad, (3) an assay platform comprising a substrate with at least one test region having a test reagent immobilized thereon, the test reagent being specific to the analyte, and (4) an optional absorbent pad. The conjugate reagent typically is labeled.

When the biological sample (e.g. vitreous sample) is delivered to the test chamber and contacts the sample pad, the arrangement is such that the fluid in the sample flows from the sample pad to the conjugate pad to the assay platform by capillary action. In typical embodiments, the conjugate reagent and test reagent are antibodies or aptamers. The reagents may bind to different epitopes of the analyte or to the same epitope (generally the case for competitive type assays).

The assay platform optionally includes a control region having a control binding reagent immobilized thereon, wherein the control binding reagent specifically binds to the at least one conjugate reagent. In an alternative embodiment, the assay platform is configured to detecting two or more different analytes in the sample. For example, the assay platform may include a first test region with a first test reagent specific to a first analyte and a second test region with a second test reagent specific to a second analyte. In addition, the conjugate pad may be a single pad with two or more different conjugate reagents pertaining to the different analytes, or may involve two or more different conjugate pads each loaded with a separate conjugate reagent.

Another embodiment is a point-of-care system for detecting an analyte in a sample (e.g. vitreous humor) that includes a sample acquisition device. Preferably, the sample acquisition device has a cannulated needle with an aspiration inlet. The sample acquisition includes an aspiration conduit that is, removably or permanently in fluid communication with the aspiration inlet and an aspirator. The system further includes an analyte detection device that is in fluid communication with the aspiration conduit and the aspirator, whereby fluid acquired through the aspiration inlet is delivered to the analyte detection device for analysis.

Examples of an analyte detection device useful with system embodiments discussed herein can include a microfluidics device or a lateral flow immunoassay (also referred to herein as LFA or LFIA) device. In a typical embodiment, the analyte detection device includes a lateral flow assay device that has (1) a sample pad onto which an analyte-containing sample to be tested is provided, (2) a conjugate pad onto which a conjugate reagent is loaded, (3) an assay platform, and (4) an absorbent pad, all disposed on a backing. Assay platforms typically are a membrane in operable contact with the sample pad and/or conjugate pad and adsorbent pad, and include (1) a region (e.g., spot or stripe) onto which a analyte reagent specific to the analyte of interest (e.g. test line stripe) is bound for capturing the analyte and (2) a region onto which a control reagent is bound (e.g. control line stripe). General background information regarding lateral flow immunoassay systems is provided in Lateral Flow Immunoassay, Raphael C. Wong and Harley Y. Tse (Editors), 2009, Humana Press, a part of Springer Science+Business Media, LLC. (Library of Congress Control Number 2008939893) and U.S. Pat. No. 8,011,228.

A non-limiting list of examples of microfluidics devices include those taught in Toward one-step point-of-care immunodiagnostics using capillary-driven microfluidics and PDMS substrates, Luc Gervais and Emmanuel Delamarche, Lab Chip, 2009, 9:3330; Gordon and Michel, Clinical Chemistry, April 2012 58(4):690-698; Chin et al., Lab Chip, 2012, 12, 2118-2134; Chin et al., Nature Medicine (2011) 17:1015-1019. The analyte detection device optionally is adapted such that the acquired fluid sample is received by the analyte detection device and transferred to a sample receiving zone of a microfluidic device associated with the analyte detection device. Typically, the analyte detection device includes a sample staging chamber for receiving the sample and a test chamber in which the microfluidic device resides. The sample staging chamber is typically in fluid communication with the aspiration conduit and the aspirator, as well as the test chamber.

In a typical embodiment, the analyte detection device includes a housing having an inlet connected (e.g. removably or permanently attached or integral) to the aspiration conduit and/or an outlet connected to the aspirator. The housing also optionally can include a window allowing visibility into the test chamber. The analyte detection device may further include a light for illuminating the assay platform. The light may be implemented in a fashion such that the label of the labeled conjugate reagent is detectable on the assay platform.

In an alternative embodiment, the analyte detection device also can include a sensor that is implemented to sense the presence of label on the assay platform. The sensor typically is one that can detect electromagnetic radiation. For example, the sensor may detect a radiation from a radioactive isotope label, fluorescence from a fluorescent label, color amount/intensity from a color signal (e.g. such as red color produced from gold label or horseradish peroxidase), etc.

In one embodiment, the aspirator can be a syringe or pump. The aspirator can be connected to the aspirator outlet of the housing as described above. In an alternative embodiment, the aspirator can be at least partially contained within the housing such that it can be actuated by the user thereby supplanting the need for an aspiration outlet on the outside of the housing.

In a further embodiment, the cannulated needle used in the sample acquisition device includes an elongated body having a tapered distal tip. Typically, the tapered distal tip is sharp, to assist with entry into tissue. The aspiration inlet may be at the tapered distal tip, but typically is upstream so as to be positioned on the elongated needle body. In the context of obtaining eye fluid samples, particularly samples of vitreous humor or other viscous or gelled material, the acquisition device also can include a cutting mechanism to assist with obtaining the sample. Examples of an acquisition device having a cutting mechanism include, but are not limited to, INTRECTOR® or RETRECTOR® (Insight Instruments™) systems, or those described in U.S. Pat. No. 5,487,725; 5,716,363; 5,989,262; 6,059,792; 7,549,972; 8,216,246; or 8,608,753. The aspiration inlet preferably receives fluid which flows into the aspiration conduit wherein a portion of the conduit resides within the elongated needle body and courses out of the acquisition device to another portion typically in the form of flexible tubing. The aspiration conduit is directly or indirectly connected to the analyte detection device.

While the specific acquisition device embodiments disclosed herein are particularly adapted for acquisition of eye fluid samples, those skilled in the art will appreciate that acquisition devices designed for acquisition of other types of samples, e.g. blood, saliva, semen, urine, tears, spinal fluid, embryonic fluid, or vaginal fluid may be implemented.

According to a further embodiment, a lateral flow immunoassay (LFIA) strip is provided that includes a sample pad for receiving a sample containing at least one analyte, wherein the analyte comprises an angiogenic ocular analyte or an inflammatory ocular analyte, or both. The LFIA also includes at least one conjugate pad in operable contact with the sample pad. The at least one conjugate pad may be loaded with a first antibody comprising first antibody specific against the at least one ocular analyte and tagged with detectable label or a second antibody comprising a second antibody specific against the at least one inflammatory ocular analyte tagged with a detectable label. Also included is an assay platform in operable contact with the at least one conjugate pad, the assay platform comprising a substrate having a test region onto which first test antibody directed to the at least one analyte is immobilized and optionally a control region onto which a control antibody directed to the first or second antibody is immobilized. Additionally, the LFIA strip can contain an optional absorbent pad in operable contact with the assay platform.

The sample pad, at least one conjugate pad, and the assay platform and absorbent pad preferably are arranged successively such that fluid flows from sample pad, to the at least one conjugate pad, to the assay platform and to the absorbent pad.

The at least one analyte can include an angiogenic ocular analyte and an inflammatory ocular analyte, and the LFIA can further include a second test region onto which a second test antibody directed to the at least one analyte is immobilized. Generally, the first test antibody is specific to the angiogenic ocular analyte and the second test antibody is specific to the inflammatory ocular analyte, or vice versa. In an alternative embodiment, the at least one conjugate pad includes a first conjugate pad including a first conjugate antibody and a second conjugate pad including a second conjugate antibody, wherein the first and second conjugate antibodies are specific to an ocular angiogenic analyte or an ocular inflammatory analyte, respectively, or vice versa.

Yet another embodiment is a method of detecting at least one analyte in a sample that involves obtaining a vitreous humor sample from a subject; subjecting the vitreous humor sample to an LFIA strip as described above; and detecting an amount of the at least one analyte on the test region.

A further embodiment pertains to an analyte detection device that includes (1) a housing comprising a sample staging chamber and a test chamber that comprises reagents that specifically interact with an analyte; (2) at least one portal in fluid communication with an aspiration conduit and an aspirator; and (3) at least one valve positioned between the sample staging chamber and the test chamber. The analyte detection device can include an LFIA or microfluidic device that has one or more reagents for reacting with and detecting analyte(s) of interest.

In an alternative embodiment, the device includes at least two assay platforms. For example, the device may include a first assay platform comprising a substrate having a test region onto which first test antibody directed to the at least one angiogenic ocular analyte is immobilized; and a second assay platform comprising a substrate having a second test region onto which second test antibody directed to the at least one inflammatory ocular analyte is immobilized. The first and second assay platforms may be arranged in parallel to the at least one conjugate pad. In an even more specific embodiment, analyte detection device includes an LFIA strip that includes a first conjugate pad and a second conjugate pad that are in operable contact with the first assay platform and second assay platform, respectively.

According to another embodiment, a method of determining efficacy of an agent administered to an eye is provided. The method involves obtaining a vitreous humor sample from a subject; subjecting the vitreous humor sample to a LFIA strip as described herein; detecting an amount of the at least one analyte on the test region; and correlating the amount of the at least one analyte with a predetermined level or range, wherein if the at least one analyte is at or within the predetermined level or range, respectively, the agent is determined as effective. The method may further involve administering a first dose of an agent to the eye of the subject and optionally administering a second dose if the amount of the least one analyte is outside the predetermined level or range. In a specific embodiment, the dosage amount of the second dose is adjusted based on the amount of at least one analyte.

B. Specific System Components

It has been discovered that a certain threshold level of analyte must be detected in a sample in order to provide beneficial information regarding disease state. Through significant trial and error experimentation, optimal concentrations of the conjugate antibody and coating antibody in order to properly detect the analyte, while avoiding false positives, have been determined. This information is provided below.

For single analyte detection using a lateral flow immunoassay device, the membrane of the assay platform generally includes a first test region containing an immobilized capture molecule such as an antibody or aptamer as the test reagent. The capture molecule typically is a molecule that is capable of specifically binding the target analyte in the sample. Any specific binding partner can serve as the capture molecule, however most typically an antibody or antigen binding domain thereof, or an aptamer is used. Specifically, the following non-limiting list of capture molecules are contemplated as suitable for use with the inventive systems, devices, assays and methods: polyclonal antibodies or antigen-binding fragments thereof, monoclonal antibodies or antigen-binding fragments thereof, scFv, sdAb, aptamers, affibodies, affimers, avimers, aptides, peptides, oligomer nucleic acids, and the like, so long as the capture molecule is specific to the analyte of interest and is able to bind thereto specifically and with high affinity.

The capture molecule or capture antibody (also referred to herein as the membrane capture molecule, membrane antibody, coating molecule or coating antibody in the context of LFIA) is a capture molecule that is immobilized to the assay platform at a certain location, preferably on or in a test line. To avoid interference with the binding to the analyte, the conjugate reagent is a conjugate antibody (or other specific binding molecule) and recognizes a different epitope than the capture molecule recognizes. In certain embodiments, the conjugate binding molecule typically is a monoclonal antibody or binding fragment thereof which is has highly specific binding (avidity and affinity) for the target analyte. The test reagent typically is an antibody (either polyclonal or monoclonal) or aptamer, or any specific binding partner that binds to the target.

In typical embodiments, the device is in a direct or double antibody sandwich format, in which case the conjugate reagent is specific for a first epitope or area on the target analyte, and the capture molecule is specific for a second epitope or area on the target analyte different from the first epitope and non-overlapping and/or non-interfering with the binding of the conjugate reagent to the target.

In an alternative embodiment, the assay device is in a competitive reaction scheme format, where the first capture antibody is adsorbed or otherwise immobilized on the membrane and has an antigen or other specific binding partner that can be recognized by a second antibody or specific binding partner. In this embodiment, the first assay region having the first capture molecule is placed on the membrane at a distance from the conjugate pad. Such distance will depend on the device geometry, and may for example be in the range of 10-50, or 15-35 mm, from the conjugate pad, or at least 10, 15, 20, or 25 mm from the conjugate pad. The first test region is usually a line and may be of any suitable width, and determination of such parameter is well known in the art. Example widths include 0.5, 0.8, 1, 1.3, 1.5, or 2 mm.

The membrane further comprises an absorbed, immobilized second assay region having a control capture molecule different from the first capture molecule. In typical embodiments, the device is in a direct or double antibody sandwich format, in which case the control capture molecule is specific for the conjugate molecule or antibody. In alternative embodiments, the device is in a competitive reaction scheme format where binding of the analyte with the conjugate molecule interferes with binding of the capture molecule, thus a lack of binding to the first conjugate molecule and binding to the control molecule indicates a positive signal.

As indicated, the second assay region typically functions as a control region, and is configured to indicate that the test is properly completed, for example that conjugate has reached the second test region. In such embodiments, the first test region operates as the test line to indicate the presence or absence of analyte in the sample. The first test region is positioned between the conjugate pad and the control test region. The amount of space between the test region and control region may vary according to the device geometry, but will typically be within the range of 5-30 or 10-20 mm, or will be at least 5, 10, or 15 mm. The test region and control regions collectively differentiate between analyte-bound and analyte-unbound conjugate.

In certain embodiments, the assay platform is configured for multiplex detection of at least two analytes. Configurations for multiplexed LFIA devices are known in the LFIA art. In embodiments of the multiplex device, the device comprises a second test region of a second capture molecule different from the first capture molecule and control molecule reagents, wherein the second capture molecule is specific for the second analyte of the sample to be detected. This second test region can be positioned between the first test region and the control region, wherein the first test region remains closest to the conjugate pad and the control region remains furthest from the conjugate pad. In such an arrangement, the control region can function as a control line for both first and second test regions. In additional embodiments, the device may further comprise additional test regions containing capture antibodies to other analytes.

The absorption pad of a typical device is a means for physically absorbing the fluid sample which has chromatographically moved through the chromatography medium (e.g., via capillary action) and for removing unreacted substances. Thus, the absorption pad is located at the end of the lateral flow assay strip to control and promote movement of samples and reagents and acts as a pump and container for accommodating them. The speeds of samples and reagents may vary depending on the quality and size of the absorption pad. Commonly used absorption pads are formed of water-absorbing material such as cellulose filter paper, non-woven fabric, cloth, or cellulose acetate.

One embodiment provides an LFIA device that includes (1) a sample pad, (2) a conjugate pad in operable contact with the sample pad and at least one conjugate reagent specific to an ocular analyte loaded thereon, (3) an assay platform including a substrate with at least one test region having a test reagent immobilized thereon, the test reagent being specific to the analyte, and (4) an optional absorbent pad. In a specific embodiment, the sample pad, conjugate pad, assay platform and absorbent pad are disposed on a common backing material, typically as a strip. In a more specific embodiment, the LFIA device includes at least a first test region and at least a second test region, wherein the first test region has an antibody-specific angiogenic ocular analyte and the second test region has an antibody-specific inflammatory ocular analyte, or vice versa. In a specific embodiment, the angiogenic ocular analyte is a growth factor such as VEGF and the inflammatory ocular analyte is an interleukin such as IL-6 or IL-8.

C. Reagents

Any molecule that can specifically bind to the target analyte or an epitope or other portion thereof is contemplated for use with the invention as a capture molecule to bind the target for detection or as a detection reagent, for example. For example, the assays can take advantage of the ability to specifically bind a particular partner of antibodies (including monoclonal antibodies, polyclonal antibodies, multispecific/bispecific antibodies, Fab fragments, Fd fragments, Fv fragments, dAb fragments, F(ab′)₂ fragments, single chain Fv fragments, and the like), aptamers (including peptide, DNA and RNA aptamers), or any other

### Antibodies

Antibodies for use with the invention include polyclonal antibodies raised to the target antigen or a portion or hapten thereof, however monoclonal antibodies are preferred. The term “monoclonal antibody” refers to an antibody molecule obtained from a substantially homogenous population of antibodies. Monoclonal antibodies can be produced, for example, according to any known method in the art, including the traditional hybridoma methods of Kohler and Milstein, Nature, 256:495, 1975 or recombinant methods according to Cabilly et al., U.S. Pat. No. 4,816,567 or Mage and Lamoyi, Monoclonal Antibody Production Techniques and Applications, pages 79-97. Marcel Dekker Inc., New York, 1987.

Antibodies specific against the ocular analytes useful in the embodiments discussed herein are known in the art and are commercially available. Companies that supply such antibodies include Abcam™ (Cambridge, Mass.), Santa Cruz Biotech™ (Dallas, Tex.), Sigma Aldrich™ (St. Louis, Mo.), Cell Signaling Technology™ (Danvers, Mass.), R&D Systems™ (Minneapolis, Minn.), Novus Biologicals™ (Littleton, Colo.) and Life Technlogies™ (Carlsbad, Calif.), inter alia. Conjugate antibodies specific against IL-6 include, but are not limited to, monoclonal anti-human IL-6 antibody or alternatively, polyclonal anti-human IL-6 antibody (e.g. goat-anti-human IL-6, rat-anti-human IL-6, or rabbit-anti-human IL-6). An example of an anti-IL6 antibody includes ab6672 (Abcam™). In certain embodiments, VEGF conjugate antibodies may include, but are not limited to, VEGF M1 or M2, Avastin (Genentech™, Inc. San Francisco, Calif.)), EYLEA (Regeneron™, Tarrytown N.Y.), and Lucentis (Genentech™, Inc.). VEGF coating antibodies may include VEGF P1, P2 or P3, VEGF RαH RD system or VEGF GαH PoeroTech. An example of an anti-MCP-1 antibody includes ab9669 (Abcam™). The above antibodies are commercially available. An example of a SPARC antibody includes ab61383 (Abcam™). United States Patent Publication No. 2010/0150920 and WO/2008/156752 are cited for information concerning ocular analytes and antibodies that can be used for detection.

### Aptamers

An aptamer is a small single-stranded nucleic acid (DNA or RNA) or a short variable peptide domain, attached at both ends to a protein scaffold in a loop. The binding structures are selected from a large random-sequence pool (or are found naturally in riboswitches) and fold into a well-defined three-dimensional structure that specifically binds with high affinity to a specific target molecule. Aptamers can be selected for specific binding to any molecular target, including proteins, peptides, and the specific biomarkers identified herein. Aptamers bind with high specificity and affinity, and can bind strongly. Upon recognition of their target, aptamers bond by internal complementary RNA base pairing. This base pairing creates secondary structures such as short helical arms and single stranded loops. See Tuerk and Gold, Science 249:505, 1990; Ellington and Szostak, Nature 346:818, 1990; Eaton, Curr. Opin. Chem. Biol. 1:10-16, 1997; Famulok, Curr. Opin. Struct. Biol. 9:324-9, 1999, and Hermann and Patel, Science 287:820-5, 2000 for further description of RNA and DNA based aptamers.

RNA aptamers can form diverse complex secondary and tertiary structures that bind the target with the entire sequence. Production of RNA aptamers requires reverse transcription, in which RNA is converted into DNA during their synthesis by SELEX, a step not necessary for DNA aptamers. DNA aptamers also form complex secondary and tertiary structures that bind the target with the entire sequence, but the possible three-dimensional structures are somewhat less diverse than RNA aptamers.

Nucleic acid aptamers typically are about 15-60 nucleotide bases long, but can be shorter or longer, including up to 200 nucleotides or more. They are most commonly generated using a combinatorial chemistry procedure termed “systematic evolution of ligands by exponential enrichment” (SELEX). The term “SELEX” refers to a combination of selecting nucleic acids that interact with a designated target molecule in the desired manner, usually by high affinity binding to the target, and amplification of those selected nucleic acids. This method identifies the nucleotide sequences (aptamers) that have the desired binding characteristics.

SELEX (a method for in vitro evolution of nucleic acids for the desired binding characteristics) involves preparing a large number of (usually randomized) candidate nucleic acids and binding a mixture of these candidates to the desired target, washing to remove unbound material, separating the bound nucleic acids, and isolating and identifying the bound sequences. These purified individual sequences are the aptamers. Usually, several rounds of selection are performed to refine and improve the affinity of the selected aptamer, usually alternating with rounds of amplification of the sequences. Thus, starting with a randomized mixture, repeated cycles of contacting with the target under binding conditions, purifying bound sequences and amplifying the bound sequences, SELEX results in a ligand-enriched mixture of nucleic acids which can be repeated as many times as needed to yield a highly specific, strong-binding nucleic acid aptamer. This process is described in more detail in U.S. Pat. Nos. 5,475,096, 5,580,737, 5,567,588, 5,705,337, 5,707,796, 5,763,177, 6,011,577, and 6,699,843. Another screening method to identify aptamers is described in U.S. Pat. No. 5,270,163. Any of the methods described in these patents can be used to produce aptamers suitable for this invention.

Bead-Based Methodology—

DNA aptamers may also be generated via an approach which does not involve a SELEX-based methodology. In such an approach a combinatory library of up to 10¹⁵ different DNA molecules is created. Library members are conjugated to a bead, with each bead harboring only a single species of DNA. A single round of bind is performed and the beads which bind the analyte have their DNA aptamer sequenced. The advantage of a bead-based approach is that it is amenable to the use of non-natural DNA bases which through the introduction of novel side chains offer an enhanced binding repertoire. The SELEX approach typically does not work with unnatural bases as they cannot be amplified in a polymerase chain reaction.

This same SELEX process can be used to select aptamers that have improved characteristics, including, but not limited to higher affinity or avidity, improved stability and the like. In addition, the aptamers can be modified as described in U.S. Pat. Nos. 5,660,985 and 5,580,737, using SELEX or photoSELEX procedures. In particular, SELEX can be used to identify aptamers that have desirable off-rate characteristics. See U.S. Patent Publication Nos. 2009/0004667 and 2009/0098549, which provides methods for improving (slowing) the disassociation rates for selected aptamers.

Peptide aptamers are short peptide sequences, usually about ten to twenty amino acids in length, attached as a loop (at both ends) to a protein scaffold. The scaffold can be any protein which is sufficiently soluble and compact. The bacterial protein thioredoxin-A is commonly used, with the variable loop inserted within the reducing active site (a -Cys-Gly-Pro-Cys- loop) in which the two cysteine residues can form a disulfide bridge.

Because peptide aptamers are small, simple peptides with a single variable loop region tied to a protein at both ends, the peptide aptamer tertiary structures are constrained by the protein scaffold to which they are attached, reducing flexibility and often therefore effectiveness. This structural constraint also, however, can greatly increase the binding affinity of a peptide aptamer to levels comparable to an antibody's (nanomolar) range.

Peptide aptamers that bind with high affinity and specificity to a target protein can be isolated by a variety of techniques known in the art. Peptide aptamers can be isolated from random peptide libraries by yeast two-hybrid screens as described in Xu et al., Proc. Natl. Acad. Sci. 94:12,473-12,478, 1997 or by ribosome display as described in Hanes et al., Proc. Natl. Acad. Sci. 94:4937-4942, 1997. They also can be isolated using biopanning and surface display technology, for example from combinatorial phage display libraries, mRNA display, ribosome display, bacterial display, yeast display, or chemically generated peptide libraries. See Hoogenboom et al., Immunotechnology 4:1-20, 1998. For example, small peptides can be displayed on a scaffold protein (e.g., one based on the FKBP-rapamycin-FRB structure and selected based on interactions between the peptides and the desired target molecule, controlled by the small molecule, rapamycin, or by non-immunosuppressive analogs. This process is known as “selection of ligand regulated peptide aptamers (LiRPAs).

Aptamers have some advantages over antibodies, including the wider range of possible targets due to their ability to structurally conform to the binding site on their targets and their selection in the absence of an antigen that produces an immune response, and (for nucleic acid aptamers) the fact that they themselves do not produce an immune response. Unlike aptamers, antibodies can produce undesirable immune responses. In addition, aptamers are more stable chemically, cheaper, and easier to produce than antibodies, are more consistent lot-to-lot and require less specialized equipment. DNA and RNA aptamers also can differ in sequence and folding pattern even when selected for the same target. Aptamers, however can be limited because sometimes the non-covalent bonds they form with target molecules can be too weak to be effective (i.e., they have a weak or fast off-time). In addition, aptamers are digested by enzymes unless modified.

Aptamers can be modified, for example by combination with a ribozyme to self-cleave in the presence of their target molecule. Additional possible modifications include replacing the 2′ position of nucleotides with a fluoroamino or O-methyl group for enhanced nuclease resistance. A second addition in the form of a “mini hairpin DNA” can impart a more compact and stable structure that resists enzymatic digestion and extends its life in solution. Bridging phosphorothioates also can be added, as well as end caps to reverse polarity of the chain and linker sequences (e.g., PEG) for ease in conjugation. Adding an unnatural or modified base to a standard aptamer can increase its ability bind to target molecules as well. Further, “secondary aptamers” also are contemplated for use with the invention in certain embodiments. Secondary aptamers are designed to contain a consensus sequence derived from comparing two or more known aptamers to a given target.

### Additional Specific Binding Reagents

Avimers are artificial antibody mimetic proteins which can bind certain antigens by multiple binding sites. Avimers are made up of two or more peptides of about 30-25 amino acids each, connected by a linker. The peptides are derived from receptors for the target protein, usually the A domain of a membrane receptor, each binding to different epitopes on the same target. The multiple binding domains increases avidity for the target protein. Avimers also can be constructed with binding domains directed against epitopes on different targets, creating a bispecific antibody mimetic. Avimers generally bind to target in sub-nanomolar ranges, and are more stable chemically than antibodies. They can be produced by selecting for binding domains as is done for peptide aptamers, for example using display techniques such as phage display and panning in cycles.

Affimer molecules are small (about 12-14 kDa), highly stable recombinant proteins that mimic monoclonal antibodies by specifically binding to a selected target with two (or more) binding domains. The affimer protein is derived from the cysteine protease inhibitor family of cystatins and based on the cystatin protein fold, but can be modified with different tags and fusion proteins. Affimers contain two peptide loops and an N-terminal sequence that can be randomized and screened to discover sequences that strongly and specifically bind to a desired target, similarly to monoclonal antibodies. The peptides are stabilized by a protein scaffold that constrains the tertiary structure and thereby increases binding affinity. Affimers are stable to temperature and pH extremes, freezing and thawing, and generally have low steric hindrance compared to antibodies.

Affimers are easy to express at high yields using bacterial, mammalian, insect or any convenient cells. General methods are as follows: A phage display library of about 10¹⁰ randomized potential target-binding sequences is generated and screened to identify a sequence with the desired high affinity and specific binding. Multiple rounds of screening, purification and identification improve the characteristics of the identified molecule, and the protein sequence is generated using recombinant systems as known in the art.

Affibodies are small (generally about 6 kDa) engineered antibody-mimetic proteins originally based on the Z domain of protein A, which binds IgG. Currently, however, the scaffold for the binding site has been modified and substituted to create different surfaces. These protein scaffold molecules usually have a three alpha helix bundle structure. The binding site contains 13 randomized amino acid residues which are screened for binding to the desired target using phage display or other display technologies as described above. The affibody molecules then are expressed in a host cell, such as bacterial, mammalian or insect cells or are produced by chemical synthesis. Affibodies sometimes are fused head-to-tail to create bi- or multi-specific binding proteins. Affibodies can be produced with sub nanomolar or picomolar affinity for the target molecule, and are stable.

Aptides are high-affinity peptides described in United States Patent Publication No. 2011-0152500, where they are referred to as “bipodal-peptide binders” and in Kim et al., “Bio-inspired design and potential biomedical applications of a novel class of high-affinity peptides” Angew. Chem. Int. Ed. Engl. 51(8):1890-1894, 2012. Aptides are antibody substitutes with a stabilizing rigid linker or backbone and two short peptides which specifically bind a target. The peptides are randomized and selected for specific binding to a desired target as known in the art and described briefly herein. The linker can be a strand which forms a bound loop due to the presence of a parallel and an antiparallel strand, for example, or another structure which forms non-covalent bonds to hold the strands together to form a stable structure, preferably with a beta-hairpin motif. Aptides have been constructed with both L- and D-amino acids.

These, or any, specific binding molecules are contemplated for use with the invention as reagents that bind to the target or as labeled detection reagents, in any portion of the herein described assay system and device. Thus, the specific binding molecules can be used as a capture molecule, a substitute for a secondary antibody or binder, a labeled detection molecule, or any use in place of an antibody.

### Detection Reagents

A “label” refers to a substance, compound or particle that can be detected, particularly by visual, fluorescent, radiation or instrumental means. A label may be, for example, but not limited to, a pigment produced as a coloring agent or ink, such as Brilliant Blue, 3132 Fast Red 2R and 4230 Malachite Blue Lake, all available from Hangzhou Hongyan Pigment Chemical Company, China. The “label” may also be a particulate label, such as, but not limited to, blue latex beads or gold particles. The particulate labels may or may not be bound to a protein, depending upon if it is desired for the particles to move in the test strip or not. If the particles are to be immobilized in the test strip, the particles may be conjugated to a protein, which in turn in bound to the test strip by either physical or chemical means.

In specific embodiments, the label includes, but is not limited to, gold nanoparticles, colored latex beads, magnetic particles, carbon nanoparticles, selenium nanoparticles, silver nanoparticles, quantum dots, up converting phosphors, organic fluorophores, textile dyes, enzymes, liposomes and others. Any material that is used as a label should be detectable at very low concentrations and it should retain its properties upon conjugation with biorecognition molecules. This conjugation is also expected not to change features of biorecognition probes. Ease in conjugation with biomolecules and stability over longer period of time are desirable features for a good label. Concentrations of labels down to 10⁻⁹ M or even 10⁻¹² M are optically detectable. After the completion of assay, some labels generate direct signal (as color from gold colloidal) while others require additional steps to produce analytical signal (as enzymes produce detectable product upon reaction with suitable substrate). Hence the labels which give direct signal are preferable in LFIA because of less time consumption and reduced procedure. Here we discuss some of the above mentioned labels in brief.

In a specific embodiment, label may include molecules known to assist in amplifying a detectable signal, such as biotin/streptavidin or biotin-avidin complexes, tyramide signal amplification (TSA) in combination with our Alexa Fluor dyes, chromogenic or chemiluminescent substrates, phycobiliproteins, fluorescent microspheres, and other amplifying molecules.

D. Optional Components

The analyte detection device, in addition to having a test chamber with reagents that interact with an ocular analyte, also can include a light positioned to illuminate the assay platform and a sensor to detect light reflected from the assay platform. The analyte detection device also can include a processor, with optional memory component, to process the signal provided by the sensor to determine an amount of signal, compare the signal to stored values and/or provide qualitative or quantitative readout, which can be provided on a display component associated therewith.

In a specific embodiment, the analyte detection device includes components suitable for colloidal gold or latex label detection on an LFIA. The analyte detection device can include a LED light source illuminating the test and control lines, sensors detecting the reflected light (electromagnetic radiation), and a processor calculating the result by comparing intensity values from sensors with predetermined values, such as calibration curve, and displaying output. The illuminating device and sensor can be associated with the housing of the analyte detection device, wherein a disposable cassette, that includes, for example, an LFIA, can be developed for a certain sample and discarded. A new disposable cassette can be loaded into the analyte detection device for analyzing another sample.

Alternatively, the LED and/or sensors can be integrated into the disposable cassette. In such alternative embodiment, the analyte detection device can comprise a display, (rechargeable) battery, memory, bar code reader for patient ID, data port or wireless technology for electronic record keeping.

### 5. Examples

This invention is not limited to the particular processes, compositions, or methodologies described, as these may vary. The examples below therefore are intended to be exemplary and not limiting.

### Example 1. Lateral Flow Immunoassay (LFIA), Antibody Capture

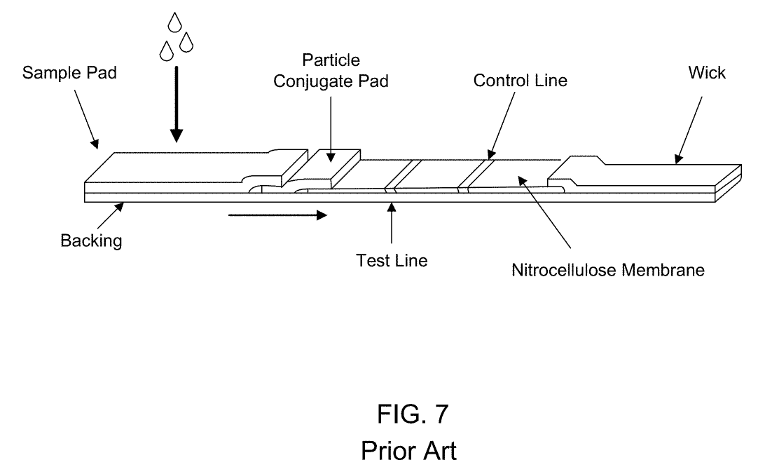

FIG. 7 shows a depiction of a typical LFIA arrangement. Sample is loaded onto a sample pad 29, which is in operable contact with a conjugate pad 31 that is loaded with conjugate antibody. A liquid, analyte-containing sample disposed on the sample pad traverses from the sample pad to the conjugate pad where the conjugate antibody binds to analyte in the sample. The analyte-containing sample (now with analyte/conjugate antibody complex) moves from the conjugate pad to the assay platform 33. The assay platform 33 typically includes at least one test region 35 having a test reagent immobilized thereon, the test reagent being specific to the analyte. As analyte-containing sample traverses across the assay platform, analyte of the analyte/conjugate antibody complex binds to the test reagent. The assay platform also includes a control region which has an antibody that is directed to conjugate antibody. This assists in determining whether the assay developed properly, and/or assists in calibrating the signal of the test region(s). The LFIA may also include an absorbent pad 37, which helps drive the flow of analyte containing sample via capillary action.

In the context of lateral flow immunoassay devices, these are typically constructed of a solid support that provides lateral flow of a sample through the assay platform when a sample is applied to a sample pad that is in operable contact with the assay platform. The sample pad and the assay platform are typically constructed of a material such as nitrocellulose, glass fiber, paper, nylon, or a synthetic nanoporous polymer. Suitable materials are well known in the art and are described, for example, in U.S. Pat. No. 7,256,053 to Hu, U.S. Pat. No. 7,214,417 to Lee et al., U.S. Pat. No. 7,238,538 to Freitag et al., U.S. Pat. No. 7,238,322 to Wang et al., U.S. Pat. No. 7,229,839 to Thayer et al., U.S. Pat. No. 7,226,793 to Jerome et al., RE39,664 to Gordon et al., U.S. Pat. No. 7,205,159 to Cole et al., U.S. Pat. No. 7,189,522 to Esfandiari, U.S. Pat. No. 7,186,566 to Qian, U.S. Pat. No. 7,166,208 to Zweig, U.S. Pat. No. 7,144,742 to Boehringer et al., U.S. Pat. No. 7,132,078 to Rawson et al., U.S. Pat. No. 7,097,983 to Markovsky et al., U.S. Pat. No. 7,090,803 to Gould et al., U.S. Pat. No. 7,045,342 to Nazareth et al., U.S. Pat. No. 7,030,210 to Cleaver et al., U.S. Pat. No. 6,981,522 to O'Connor et al., U.S. Pat. No. 6,924,153 to Boehringer et al., U.S. Pat. No. 6,849,414 to Guan et al., U.S. Pat. No. 6,844,200 to Brock, U.S. Pat. No. 6,841,159 to Simonson, U.S. Pat. No. 6,767,714 to Nazareth et al., U.S. Pat. No. 6,699,722 to Bauer et al., U.S. Pat. No. 6,656,744 to Pronovost et al., U.S. Pat. No. 6,528,323 to Thayer et al., U.S. Pat. No. 6,297,020 to Brock, U.S. Pat. No. 6,140,134 to Rittenburg, U.S. Pat. No. 6,136,610 to Polito et al., U.S. Pat. No. 5,965,458 to Kouvonen et al., U.S. Pat. No. 5,712,170 to Kouvanen et al., U.S. Pat. No. 4,956,302 to Gordon et al., and U.S. Pat. No. 4,943,522 to Eisinger et al., all of which are incorporated herein by this reference. The use of such devices for the performance of sandwich immunoassays is also well known in the art, and is described, for example, in U.S. Pat. No. 7,141,436 to Gatto-Menking et al. and U.S. Pat. No. 6,017,767 to Chandler, U.S. Pat. No. 6,372,516 to Ming Sun, all of which are incorporated herein by this reference.

As indicated above, an analyte-containing sample traverses the assay platform by way of capillary action. As the sample moves across the assay platform analytes in the sample encounter different biomolecules, such as antibodies, that bind to the analyte. Materials from which the assay platform can be made typically include, but are not limited to, nitrocellulose, glass fiber, paper, nylon, or a synthetic nanoporous polymer.

LFA reproducibility is not only influenced by design and manufacturing, but also by the components used in the assembly of the test. The assay platform membrane has a significant impact on the performance of the test results. Nitrocellulose membranes are manufactured by dissolving the raw materials in a mixture of organic solvents and water, pouring this casting mix onto a solid belt-like support, and evaporating the solvents under controlled conditions of temperature, humidity, and belt speed within the membrane manufacturing machine.

Using new membrane formulations and improved manufacturing conditions various variables of the membrane can be controlled according to techniques known in the art. Such variables include:

i. Flow Rate of Membrane This is determined empirically, and will vary according to the viscosity of the sample used. Data for the flow rates of specific membranes with specific sample types are supplied by the manufacturer.

ii. Membrane Porosity This describes the fraction of the membrane that is air (e.g., a membrane with a porosity of 0.7 is 70% air), and will have an impact on the flow rate of the membrane.

iii. Membrane Capacity By definition, this is the amount of volume of sample that a given membrane can hold, and is determined as a factor of the length (L), width (W), thickness (T), and porosity (P) of the membrane: L×W×T×P=Membrane Capacity. A second important calculation is the determination of the amount of antibody that can be bound per unit area of membrane (pertaining to the capture and control lines). This calculation involves the following variables. Dimensions of representative capture antibody line: 0.1 cm×0.8 cm=0.08 cm2. Binding capacity of membrane used for capture antibody (obtained from the membrane manufacturer).

Conjugate Pad and Reagents

In a typical embodiment, the conjugate pad comprises an absorbed but not immobilized conjugate comprising a conjugate reagent (e.g. antibody) specific for the analyte and conjugated directly to detectable marker, such as a gold nanoparticle. The conjugate reagent can be loaded onto the conjugate pad using an aqueous conjugate antibody solution. The conjugate antibody solution comprises the conjugate antibody and other components provided for solution stability, pH regulation, and the like. Examples of such ancillary components include buffers, salts, preservatives, etc. Specific examples include BSA, sucrose, trehalose, tween-20, PEG, water, HEPES, Polyvinyl pyrolidone (PVP), and the like. In an embodiment, the conjugate antibody solution is applied to the conjugate pad and allowed to dry for a period of time (e.g., 0.5, 0.7, 1, 1.5 hr) at a specified temperature (e.g., 23, 25, 30, 35, 37, 40° C.). By such application or an equivalent method, the conjugate is loaded onto the conjugate pad. In this regard, the conjugate is not immobilized on the conjugate pad, and can be carried from the conjugate pad via an assay solution, such as along a capillary flow path, into the porous membrane.

As shown in the data provided in the Examples section herein, the type of antibodies used in the assay reaction can have a dramatic impact on sensitivity as well as the accuracy of the assay (e.g. avoidance of false positives). The conjugate antibody pertains to the antibody that the sample will typically encounter first while passing along the assay platform. The conjugate antibody specifically binds to an analyte of interest in the sample. In addition, the conjugate antibody typically comprises a label associated therewith.

### Example 2. Lateral Flow Immunoassay (LFIA), Aptamer Capture

Example 1 is repeated using aptamers in place of one or more of the antibody reagents.

### Example 3. Description of Illustrated Embodiments

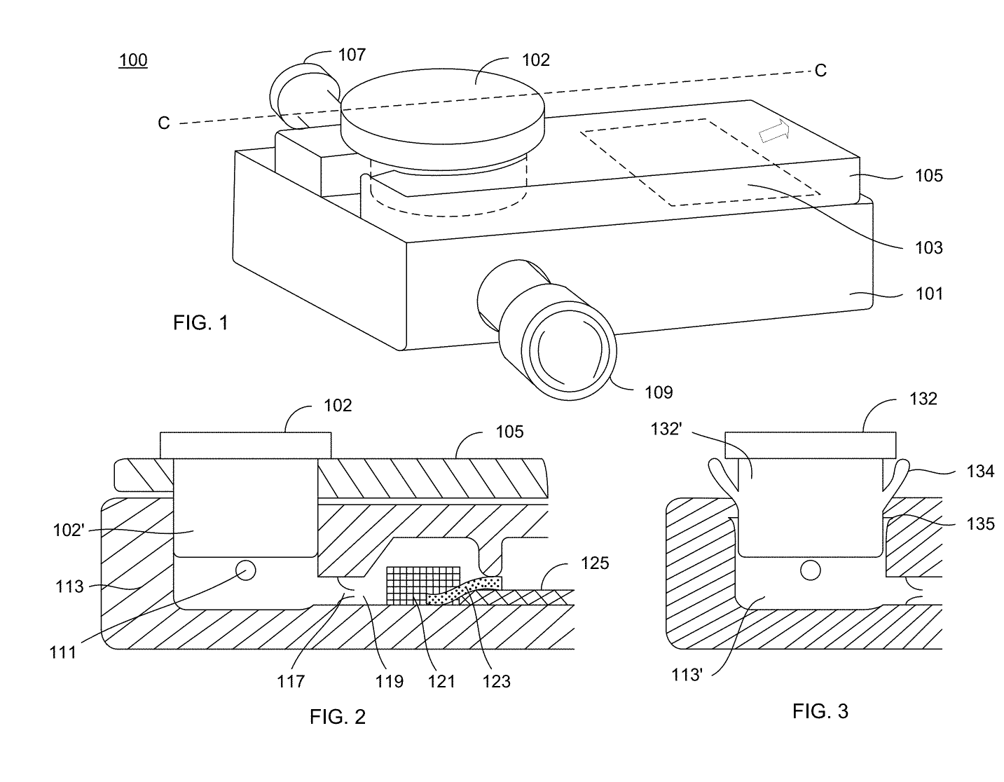

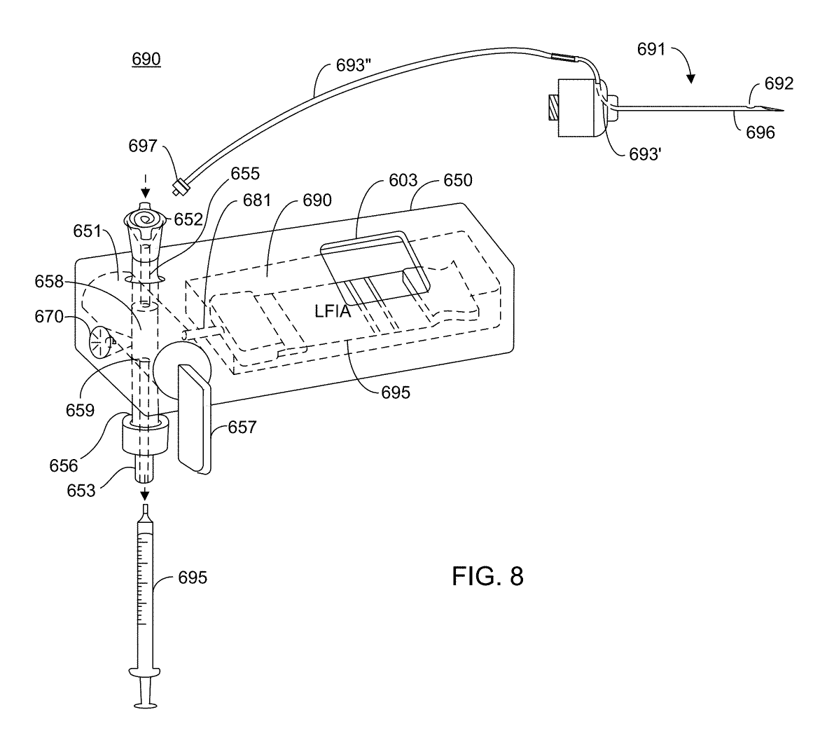

FIG. 1 shows a side perspective view of an analyte detection device 100 that receives a sample and determining the presence or amount of an analyte in the sample. The device 100 has an outer housing 101 and an inlet 107 into which a sample is delivered to the device as well as an outlet 109 to which an aspirator or other component can be connected. A sample acquisition device (such as 691 shown in FIG. 8 and described herein) can be connected to the inlet 107 and an aspirator (such as 695 shown in FIG. 8 and described herein) can be connected to 109 for purposes of delivering sample to the device 100. The device 100 includes a sample actuator 102 that serves to drive sample into internal components of the device 100. To prevent accidental actuation of the actuator 102, as well as movement of the actuator during the sample aspiration phase, a safety tab 105 overlays the top surface device but underneath a lip of the actuator 102. Shown underneath the safety tab 105 is a window 103 which allows visualization of certain components within the housing.

FIG. 2 shows a partial cross-section of device 100 along axis C-C. Safety tab 105 can be seen adjacent to the sample actuator 102. Within housing 101, a sample staging chamber 113 is provided having a portal 111 that is in fluid communication with inlet 107. The device also has a test chamber 119 that is in fluid communication with the sample staging chamber 113. A one-way valve 117 is positioned in between the sample staging chamber 113 and test chamber 119. Within the test chamber is a sample pad 121 in operable contact with a conjugate pad 123, which is in operable contact with an assay platform 125. The test chamber can be separated into different portions by a divider 124. The divider 124 helps prevent spillage of sample onto the assay platform thereby limiting movement of sample to capillary action. A sample is delivered to the sample staging chamber 113 and then pushed into the test chamber by depressing the actuator 102 (with safety tab 105 removed), which drives the lower body portion 102′ of the actuator 102 into the sample staging chamber 113.

FIG. 3 shows an alternative example of a sample staging chamber 113′ and an actuator 132. The actuator 132 includes flap portion(s) 134 which hold back the actuator 132 during sample aspiration and to prevent accidental actuation. When a threshold force is applied to actuator 132, the actuator drops downward. The walls into which the lower portion 132′ of the actuator sits may have receiving notches 135 into which the flap portion can rest upon actuation.

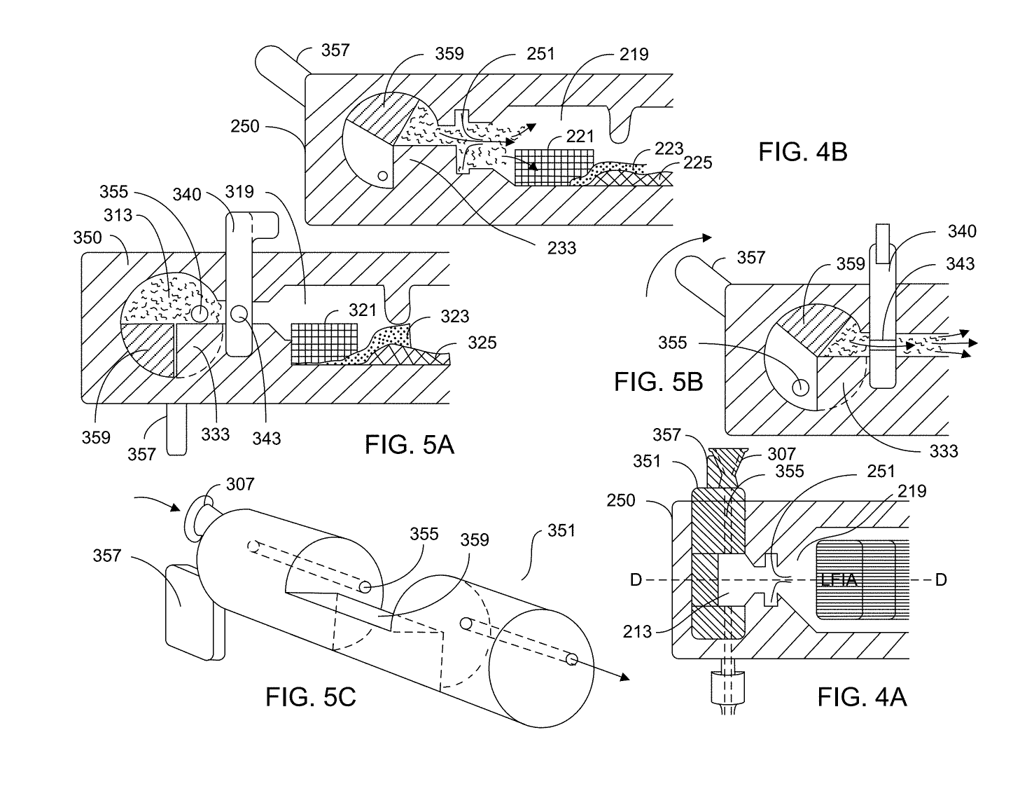

FIGS. 4A and 4B pertain to an analyte device embodiment 250. FIG. 4A represents a top, partial cross-section view that shows the interaction of sample actuator 351 that possesses a cut-away to form the sample staging chamber 213. The sample actuator 351 (see also FIG. 5C) has a channel 355 defined therein to allow sample to flow from the inlet 307 to the sample staging chamber 213. A sample acquisition device (such as 691 shown in FIG. 8 and described herein) can be connected to the inlet 307 and an aspirator (such as 695 shown in FIG. 8 and described herein) can be connected to proximally relative to channel 355 for purposes of delivering sample to the device 100. The device 250 also includes a test chamber 219 into which a test device, such as an LFIA, can be situated. A one-way valve 251 is positioned between the sample staging chamber 213 and the test chamber 219. FIG. 4B shows a partial, side longitudinal cross section along axis D-D from FIG. 4A. As shown, analyte containing sample present in the sample staging chamber 213 is transferred to the test chamber 219 in response to the rotation of the sample actuator 351 by application of force to actuator tab 357. Sample flows through valve 251 and into test chamber 219 where the fluid contacts sample pad 221. The rotation of the sample actuator serves to close the channel 355 to prevent backflow of the sample. Sample pad 221 is in operable contact with conjugate pad 223, which is in operable contact with assay platform 225. The sample staging chamber 213 is formed by the cutaway area and wedge portion 359 of the sample actuator 351 that interacts with the shelf portion 233 associated with the device housing. As the sample actuator rotates 351 the sample staging chamber 213 collapses which pushes fluid through valve 251.

FIG. 5A shows a partial side cross-section view of an analyte device embodiment 350 similar to embodiment 250 but includes a manual valve 340 that controls fluid from the sample staging chamber 313 to the test chamber 319 in place of the one-way valve 251. The manual valve 340 has passage 343 that will allow fluid in the sample staging chamber 313 to flow into the test chamber 319 when turned such as shown in FIG. 5B. The sample actuator 351 shown in FIG. 5C is the same as that shown in FIG. 4A, and is also similarly arranged such that the cutaway and wedge 359 align with shelf portion 333 to form the sample staging chamber 313. The sample actuator 351 also has a channel 355 that is in fluid communication with the sample staging chamber 313. With the valve 340 in a closed position and sample actuator 351 in fully open position as shown in FIG. 4A, sample is drawn into the sample staging chamber 313. The test chamber 319 includes a sample pad 321, conjugate pad 323 and assay platform 325. Turning to FIG. 5B, the valve 340 is opened so that the passage 343 allows the sample to flow from staging chamber to the test chamber. In conjunction with this, the actuator 351 is rotated via force to actuator tab 357, which pushes the sample through valve 340 as the wedge 359 approaches the shelf 333.

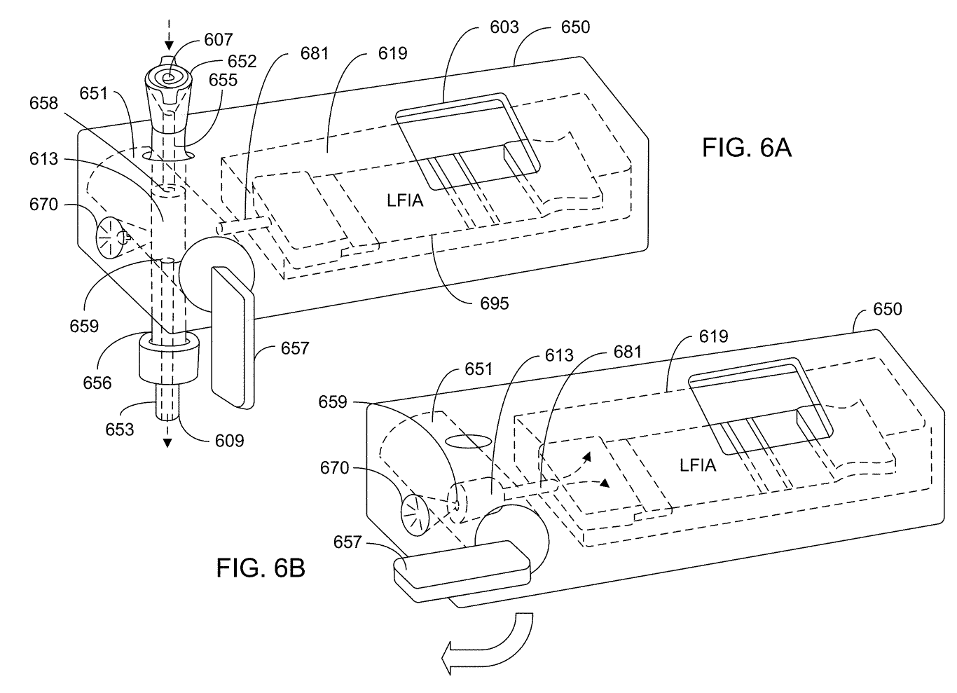

FIGS. 6A and 6B show an alternative arrangement of an analyte device 650 which receives sample from a top to bottom, or bottom to top, direction, as opposed to side to side. The sample actuator 651 has a sample staging chamber 613 that is in fluid communication with an inlet 607 via conduit portion 655 and aperture 658. In the sample receiving position, as shown in FIG. 6A, sample is delivered through inlet 607 and travels to the sample staging chamber 613. Sample is typically drawn through by aspiration applied to aspiration outlet 609 that applies a vacuum to the sample staging chamber 613 through portal 659 and conduit portion 656. In the position of the sample actuator 651 shown in FIG. 6A, the aperture 670 and test chamber conduit 681 are closed off from the sample staging chamber 613. The device further includes a test device 695 (e.g. LFIA) positioned in the test chamber 619 and a window 603 that allows visualization of at least a portion of the test device 695.

In FIG. 6B, the sample actuator 651 is rotated by turning actuator tab 657 which causes the sample chamber 613 to align so that the portal 659 aligns with aperture 670 to allow passage of air to staging chamber 613 and portal 658 aligns with the test conduit 681 to allow sample to flow to the test chamber 619.

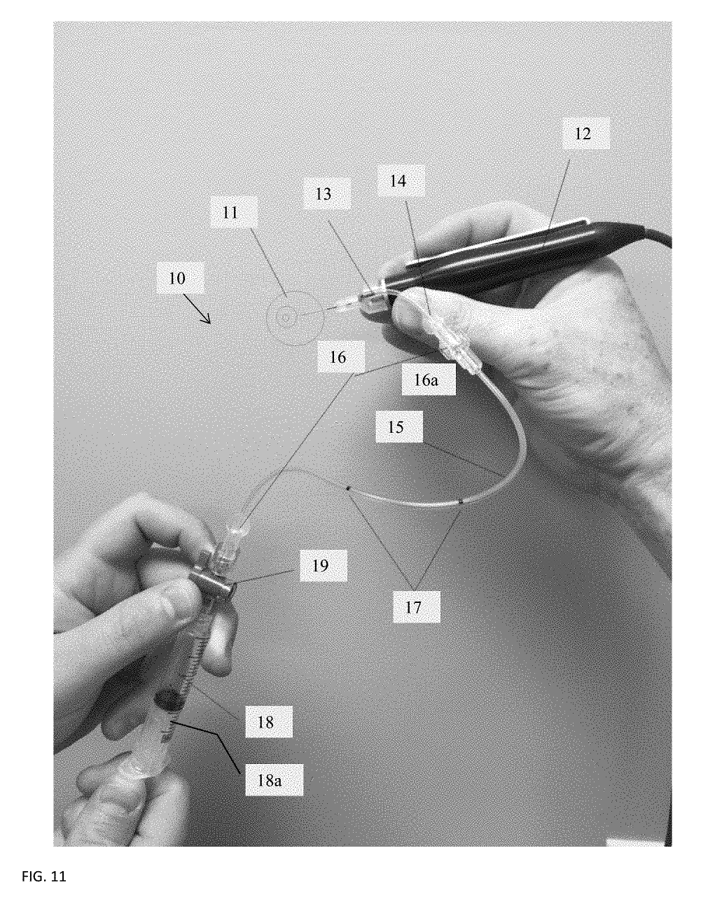

Turning to FIG. 8, shown is a system arrangement 690 a sample acquisition device (probe) 691 that comprises a cannulated needle member 696 having an aspiration inlet 692 in fluid communication with a first aspiration conduit portion 693′. The sample acquisition device 691, can be associated with a vitrectomy device (e.g. element 12 shown in FIG. 11). The first aspiration conduit portion 693′ is in fluid communication with a second aspiration conduit portion 693″. The proximal end of the second aspiration conduit portion 693″ comprises a connector 697 that engages to the distal end 652 of conduit portion 655. Also shown is an aspirator (e.g. syringe) 695 which engages to the proximal end 653 of the conduit portion 656. When engaged, the aspiration inlet 692, aspiration conduit portions 693′,693″, conduit portion 655, conduit portion 656 and aspirator 695 are all in fluid communication, and the aspiration conduit portions 693′ and 693″, conduit portion 655 and conduit portion 656 together form an aspiration conduit. The other elements shown in FIG. 8 that are not specifically discussed in this paragraph correlate to the elements discussed in relation to FIG. 6.

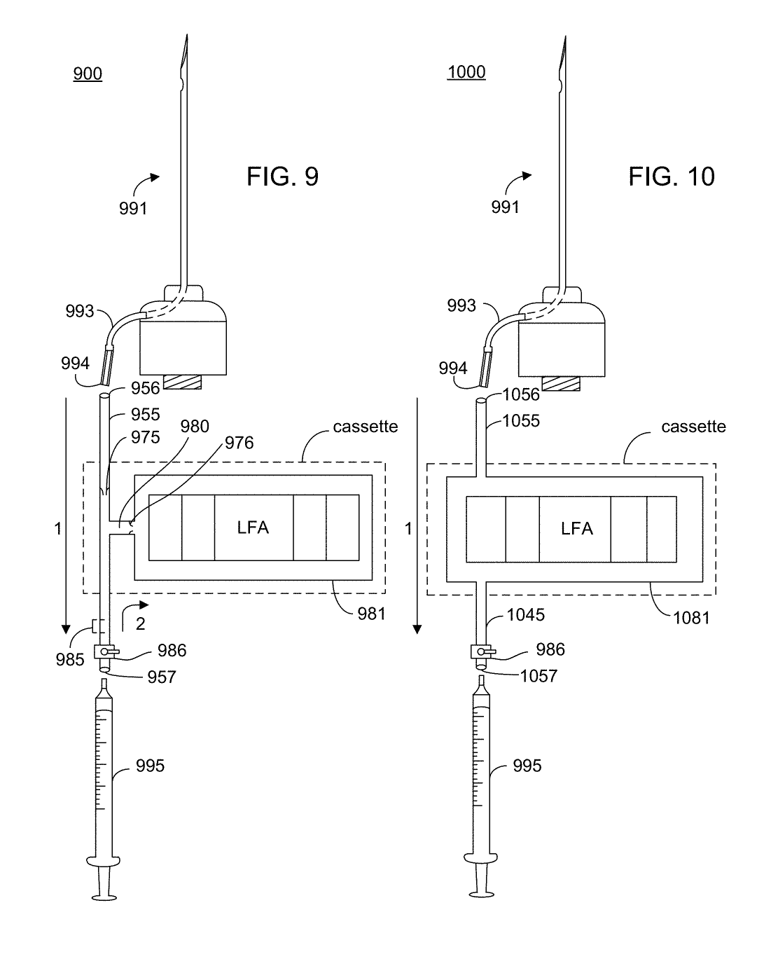

FIG. 9 shows another embodiment of a system arrangement 900. The system 900 includes a sample acquisition device (probe) 991 having an aspiration conduit portion 993 in fluid communication therewith. The sample acquisition device 991 can be associated with a vitrectomy device (e.g. element 12 shown in FIG. 11). The proximal end 994 of the aspiration conduit portion 993 is engageable to aspiration conduit portion 955 at its distal end 956. At the proximal end 957 of the aspiration conduit portion 955 an aspirator 995 (e.g. syringe) is engaged. Within the aspiration conduit portion 955 is a one-way valve 975. The aspiration conduit portion 955 is in fluid communication with the sample conduit 980 that leads to a test chamber 981, which is shown to contain an LFA strip. A one-way valve 976 is positioned at the sample conduit between the aspiration conduit portion 955 and test chamber 981.

During use of the system 900, in a first step 1, sample is drawn into the sample acquisition device 991 then through aspiration conduit portion 993 then through aspiration conduit portion 955. Provided on the proximal body of the aspiration conduit portion 955 are markings 985 to assist the user in determining the amount of sample that has been obtained. As vacuum is applied by the aspirator 995 causing fluid to flow through valve 975 while valve 976 is closed. Upon obtaining an appropriate amount of sample, as determined by sample reaching a desired marking 985, pressure is then applied by the aspirator 995 to push the sample back up the aspiration conduit 955. This causes valve 976 to open to allow passage of fluid and valve 975 to close resulting in fluid passing through the sample conduit 980 to the test chamber 981. Provided as an optional feature upstream of the proximal end 957 is a valve 986 (e.g. stopcock as shown), which may be included in the system 900 to assist in control of aspiration. A divider such as 124 shown in FIG. 2 can be provided to prevent undesired spillage or seepage of sample onto the assay platform.

Another embodiment 1000 is shown in FIG. 10, which pertains to a system arrangement that provides one directional flow for sample acquisition and analyte detection. Sample is drawn into the sample acquisition device (probe) 991 then through aspiration conduit portion 993 then through distal end 1056 of aspiration conduit portion 1055. The sample acquisition device 991 can be associated with a vitrectomy device (e.g. element 12 shown in FIG. 11). The aspiration conduit 1055 is in proximity to a LFA strip such that when sample is drawn to the test chamber 1081, by vacuum applied to proximal end 1057 of aspiration conduit portion 1045, it is delivered to the LFIA (e.g. onto the sample pad). Provided upstream of the proximal end 1057 is a valve 986 (e.g. stopcock as shown) to assist in control of aspiration. A divider such as 124 shown in FIG. 2 can be provided to prevent undesired spillage or seepage of sample onto the assay platform. The arrangement of 1000 does not require valves to achieve sample acquisition and delivery to LFA. It should be noted that the LFA strip could be substituted with a microfluidic device or like devices for detecting analytes in a sample.

Turning to FIG. 11, a further embodiment 10 is shown that is adapted for improved and facile extraction of a vitreous biopsy from a patient's eye 11. The embodiment includes a disposable vitrectomy probe 13, that is designed for operation with a surgical hand-piece (e.g. vitrectomy device) 12. The probe 13 is in fluid communication with an aspiration tube 14 that is connectable with a separate sampling tube 15. As shown, the aspiration tube 14 and sampling tube 15 are connected with conventional Luer lock connectors 16, but those skilled in the art will appreciate that other types of suitable connectors can be used for this purpose. Entry into the eye 11 is made with the sharp tipped vitrectomy probe 13.

The aspiration tube 14 and sampling tube 15 are in fluid communication with a syringe 18. Drawing the plunger of the syringe 18 creates a vacuum that pulls in the fluid (e.g. vitreous sample) through the aspiration tube and into the sampling tube 15. Further, the fill marks 17 assist with controlling the amount of vitreous sample extracted and are placed at a strategic location on the sampling tube 15 based on the minimum volume, as well as dead volume of the tube and cassette, as is discussed further below.

The extraction of vitreous humor is a very delicate process, and exceptional care must be exercised in controlling the amount removed. Because there is a vacuum build-up in the syringe, the implementation of the stopcock 19 provides immediate and fine control of the fluid extraction process.

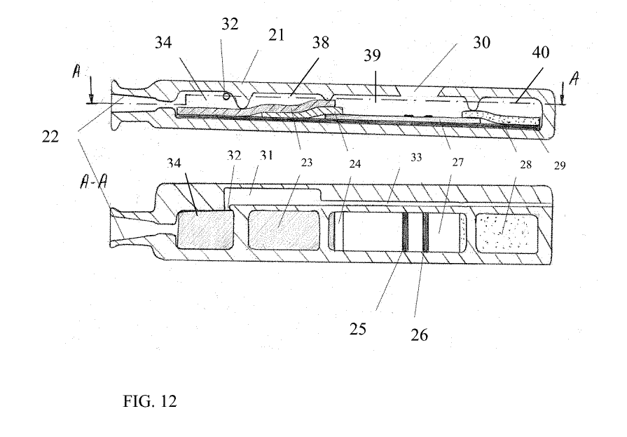

After completed sample acquisition and removal of the probe 13 from the eye 11, the sampling tube 15 is disconnected from the probe at the connector 16a, and connected to the analyte detection device (e.g. lateral flow assay cassette) 21 via connection port 22 (FIG. 12, e.g. Luer lok fitting). Focusing on FIGS. 11 and 12 (note that lower figure is a cross section of the embodiment shown in the upper figure along A-A axis), once the desired amount of vitreous sample is drawn into the sampling tube 15, as designated by fill marks 17, the sample can now be expressed into the sample staging chamber 34 of the cassette 21 by depressing the syringe plunger 18a and opening the stopcock 19. To prevent sample from flowing directly into the overflow passage 32 under gravity, the sample staging chamber 34 may be shaped conically expanding, to allow surface tension a gradual fill of the sample chamber 34 from the connection port 22. Addition of an open cell foam material may also be used for that purpose. A vent 33 in fluid connection with the sample staging chamber 34 prevents build up of excessive pressure in the sample staging chamber 34 and undesired pressurized infusion of sample into the sampling pad 23. A one way valve in the overflow passage 32 or the presence of wicking material in the overflow reservoir 31 may additionally help prevent backflow of excess sample back into the sample staging chamber 34.

Once the sample is delivered to the sample staging chamber 34, sample contacts the sample pad 23 and sample is transferred by capillary action to a sample transfer chamber 38 where a portion of the sample pad 23 contacts a conjugate pad 24. The end of the sample pad 23 and conjugate pad extend into the test chamber 39 where they contact with the assay platform 27 (e.g. membrane). Disposed on the assay platform 27 are a test region 25 and a control region 26. At the distal end of the test chamber 39 is disposed a wicking pad 28 that serves to wick sample passing along the assay platform 27. As shown the wicking pad 28 contacts the assay platform 27 at one end and is disposed within a wicking chamber 40 at an opposite end. A window 30 is also provided for visualizing the assay platform 27. As shown, the sample pad 23, conjugate pad 24, assay platform 27 and wicking pad 28 are disposed upon a backing material 29,

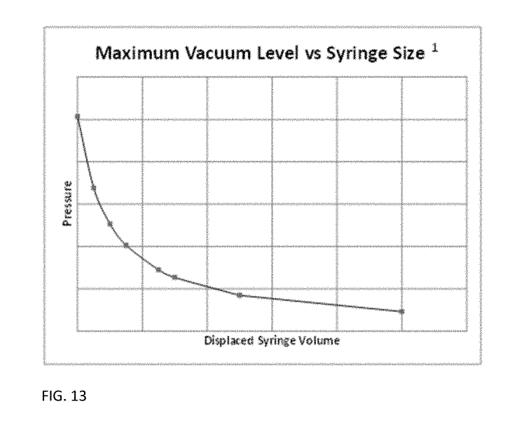

The minimum length of the sampling tube 15 is chosen to allow the assistant to operate the aspiration syringe without interfering with the surgeon's view and manipulation of the surgical instrument 12 in the operating field. The maximum length of the tube 15 is determined by the available vacuum level created by the syringe (FIG. 13) and the resulting flow rate of the sample in the sampling tube as described by the Hagen-Poiseuille equation (see Formula 1 below), as well as allowing the assistant to monitor the amount of collected fluid in the aspiration tube 14.

$$\begin{matrix}Equation 1 - {Poiseuille}^{′} s law & \\ Flow = \frac{P \times Π \times {r}^{4}}{8 \times l \times n} \\ p = preassure \\ r = radius \\ l = distance \\ n = viscosity \\ Since resistance = preassure / flow , therefore \\ Resistance = \frac{8 \times l \times n}{Π \times {r}^{4}} & Formula 1\end{matrix}$$

According to a specific embodiment, variables of certain dimensions components of the embodiment are provided for illustration purposes in reference to FIGS. 11 and 12. An overall length of the sample tube 15 of 8″ is assumed in the embodiment³. The internal diameter (ID) of the sampling tube 15 is fabricated to allow the required sample volume to take up a significant portion of the sampling tube length (assumption of 50%)⁴, with easily readable fill marks⁵ 17, which provide visual reference to the assistant when to stop the aspiration by closing the stopcock 19. The minimum sampling tube volume must take into account the dead volume of the tube 15 and cassette, not shown. For example, a sample volume of 50 ul (min of 45 ul, max of 55 uL) can thus be achieved in this embodiment by using a 0.030″ micro-bore tubing.

The sample chamber volume is equivalent to the nominal sample requirement of 35 μl for the assay⁷. Any volume in excess of the nominal sample requirement is displaced through overflow passage 32 into overflow reservoir 31. The volume of the overflow reservoir is designed to accept any overflow when the sampling tube is filled to the maximum fill mark⁸. The superscript designations in this and the preceding paragraphs refer to the noted information in Table 1.

TABLE 1

| | | |
|---|---|---|
| V_{T} | Volume in Sample Tube | |
| V_{Tmin} | Volume in Sample Tube @ min Fill Mark | |
| V_{Tmax} | Volume in Sample Tube @ max Fill Mark | ⁵⁾ V_{Tmin} + 10 ul |
| L_{T} | Length of Sample Tube | ³⁾ 8″ = 200 mm |
| L_{Tmin} | Length of Sample Tube @ min Fill Mark | ⁴⁾ 50% of L_{T} |
| L_{Tmax} | Length of Sample Tube @ max Fill Mark | |
| Dₜ | Inside Diameter of Sample Tube | |
| Vₛ | Volume of Sample required for Assay | ⁷⁾ 35 ul = 35 mm³ |
| V_{D} | Dead Volume of Tube and Cassette | ⁶⁾ 10 ul = 10 mm³ |
| V_{R} | Volume of Overflow Reservoir ⁸⁾ | |

The equations below serve to define the different parameters of Table 1. The superscript 8 in equation (3) is a cross reference to note 8 in Table 1.

Equations:

$${V}_{Tmin} \geq {V}_{S} + {V}_{D} ( 1 )$$

$${V}_{T} = \frac{π}{4} {D}_{T}^{2} {L}_{T} ( 2 )$$

$${V}_{R} \geq {V}_{Tmax} - {{V}_{S} ( 3 )}^{8}$$

$${L}_{Tmax} / {L}_{Tmin} = {V}_{Tmax} / {V}_{Tmin} ( 4 )$$

Calculations:

$$using ( 1 ) \text{:} {V}_{Tmin} \geq {V}_{S} + {V}_{D} = 35 {mm}^{3} + 10 {mm}^{3} = \underline{45 {mm}^{3}}$$

$${using}^{4} \text{:} {L}_{Tmin} = 0.5 \cdot 200 mm = \underline{100 mm}$$

$$using ( 2 ) \text{:} {D}_{T} = \sqrt{\frac{4 {V}_{Tmin}}{π {L}_{Tmin}}} = \sqrt{\frac{4 \cdot 45 {mm}^{3}}{π \cdot 100 mm}} = \underline{.76 mm} ( {.030}^{″} )$$

$${using}^{5} \text{:} {V}_{Tmax} = {V}_{Tmin} + 10 {mm}^{3} = 45 {mm}^{3} + 10 {mm}^{3} = \underline{55 {mm}^{3}}$$

$$using ( 4 ) {L}_{Tmax} = \frac{55 {mm}^{3}}{45 {mm}^{3}} \cdot 100 mm = \underline{122 mm}$$

$$using ( 3 ) \text{:} {V}_{R} \geq {V}_{Tmax} - {V}_{S} = 55 {mm}^{3} - 35 {mm}^{3} = 20 {mm}^{3}$$

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the preceding definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, any means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

### 6. References

All publications mentioned below and throughout the application are hereby incorporated by reference in their entirety.
1. Wong and Tse (Eds.) Lateral Flow Immunoassay, 2009, Humana Press, a part of Springer Science+Business Media, LLC. (Library of Congress Control Number 2008939893).
2. U.S. Pat. No. 8,011,228.
3. United States Patent Application No. 2014/0193840 A1.
4. Gervais and Delamarche, Toward one-step point-of-care immunodiagnostics using capillary-driven microfluidics and PDMS substrates. Lab Chip 9:3330, 2009.
5. Gordon and Michel. Clin. Chem. 58(4):690-698, 2012.
6. Chin et al. Lab Chip 12:2118-2134, 2012.
7. Chin et al. Nat. Med. 17:1015-1019, 2011.
8. U.S. Pat. No. 5,487,725.
9. U.S. Pat. No. 5,716,363.
10. U.S. Pat. No. 5,989,262.
11. U.S. Pat. No. 6,059,792.
12. U.S. Pat. No. 7,549,972.
13. U.S. Pat. No. 8,216,246.
14. U.S. Pat. No. 8,608,753.
15. Jones et al., Nature 321:522-525, 1986.
16. Riechmann et al., Nature 332:323-329, 1988.
17. Presta, Curr. Opin. Struct. Biol. 2:593-596, 1992.
18. Vaswani and Hamilton, Ann. Allergy, Asthma & Immunol. 1:105-115, 1998.
19. Harris, Biochem. Soc. Transactions 23:1035-1038, 1995.
20. Hurle and Gross, Curr. Opin. Biotech. 5:428-433, 1994.
21. European Patent No. 404,097.
25. International Patent Application WO 1993/01161.
26. Hudson et al., Nat. Med. 9:129-134, 2003.
27. Hollinger et al., PNAS USA 90: 6444-6448, 1993.
28. Kohler and Milstein, Nature, 256:495, 1975.
29. U.S. Pat. No. 4,816,567.
30. Mage and Lamoyi, Monoclonal Antibody Production Techniques and Applications, pages 79-97. Marcel Dekker Inc., New York, 1987.
31. Methods, Volume 38, Issue 4, April 2006, Pages 324-330, Methods for Analyzing Cytokines; Edited By Daniel G. Remick and Jill Granger, Assays for cytokines using aptamers.
32. Jo et al., VEGF-binding aptides and the inhibition of choroidal and retinal neovascularization. Biomaterials 35(9):3052-3059, 2014.

What is claimed is:

1. A system for detecting an analyte in a vitreous humor or aqueous humor sample, the system comprising
a vitreous humor or aqueous humor acquisition device comprising a cannulated needle with an aspiration inlet;
an aspiration conduit in fluid communication with the aspiration inlet;
an aspirator for applying a vacuum to the aspiration conduit; and
an analyte detection device in fluid communication with the aspirator and the acquisition device.

2. The system of claim 1, wherein the analyte detection device comprises a sample staging chamber and a test chamber that comprises reagents that specifically interact with the analyte.

3. The system of claim 2, wherein the sample staging chamber is in fluid communication with the aspirator and the aspiration conduit, and wherein the analyte detection device further comprises a valve positioned between the sample staging chamber and the test chamber.

4. (canceled)

5. The system of claim 2, wherein the test chamber comprises a sample pad, a conjugate pad comprising at least one conjugate reagent specific to the analyte loaded thereon, an assay platform comprising a substrate with at least one test region having a test reagent immobilized thereon, the test reagent being specific to the analyte; and an optional absorbent pad; wherein the conjugate reagent is labeled;
wherein the vitreous sample contacts the sample pad such that fluid in the vitreous humor or aqueous humor sample flows from the sample pad to the conjugate pad to the assay platform by capillary action;
wherein conjugate reagent and test reagent are antibodies or aptamers that bind to different epitopes of the analyte or a common epitope of the analyte and
wherein the assay platform comprises a first test region with a first test reagent specific to a first analyte and a second test region with a second test reagent specific to a second analyte.

6. (canceled)

7. (canceled)

8. (canceled)

9. (canceled)

10. The system of claim 5, wherein the assay platform comprises a control region having a control binding reagent immobilized thereon, wherein the control binding reagent specifically binds to the at least one conjugate reagent.

11. (canceled)

12. (canceled)

13. (canceled)

14. The system of claim 2, wherein the analyte detection device comprises a housing comprising an inlet connected to the aspiration conduit and an outlet connected to the aspirator.

15. The system of claim 10, wherein the housing further comprises a window allowing visibility into the test chamber; and optionally wherein the analyte detection device further comprises a light for illuminating the assay platform.

16. (canceled)

17. (canceled)

18. A lateral flow immunoassay (LFIA) strip, comprising:
a sample pad for receiving a sample containing at least one analyte, wherein the analyte comprises an angiogenic ocular analyte or an inflammatory ocular analyte, or both;
at least one conjugate pad in operable contact with the sample pad, the at least one conjugate pad loaded with a first antibody or first aptamer comprising first antibody or aptamer specific against the at least one ocular analyte and tagged with detectable label or a second antibody or aptamer comprising an second antibody or aptamer specific against the at least one inflammatory ocular analyte tagged with a detectable label;
an assay platform in operable contact with the at least one conjugate pad, the assay platform comprising a substrate having a test region onto which first test antibody or aptamer directed to the at least one analyte is immobilized and optionally a control region onto which a control antibody directed to the first or second antibody or aptamer is immobilized; and
optionally, an absorbent pad in operable contact with the assay platform.

19. (canceled)

20. (canceled)

21. (canceled)

22. A method of detecting at least one analyte in a sample, the method comprising:
obtaining a vitreous humor or aqueous humor sample from a subject;
subjecting the vitreous humor sample to a LFIA strip according to claim 18; and
detecting an amount of the at least one analyte on the test region.

23. An analyte detection device comprising:
a housing comprising a sample staging chamber and a test chamber that comprises reagents that specifically interact with an analyte;
at least one portal in fluid communication with an aspiration conduit and an aspirator; and
at least one valve positioned between the sample staging chamber and the test chamber;
wherein the device comprises at least one LFIA strip according to claim 18.

24. (canceled)

25. (canceled)

26. (canceled)

27. (canceled)

28. (canceled)

29. (canceled)

30. (canceled)

31. (canceled)

32. (canceled)

33. The system of claim 1, wherein analyte detection device comprises a housing having a test chamber that comprises reagents that specifically interact with the analyte; the test chamber comprising a first portal in fluid communication with the aspiration conduit for receiving sample from the sample acquisition device and a second portal in fluid communication with the aspirator; wherein the test chamber comprises a lateral flow immunoassay strip having a sample pad and the first portal is proximate to the sample pad such that sample is delivered thereon during aspiration.

34. (canceled)

35. (canceled)

36. A system for detecting an analyte in a vitreous humor or aqueous humor sample, the system comprising
a vitreous humor or aqueous humor acquisition device comprising a cannulated needle with an aspiration inlet;
an aspiration tube in fluid communication with the aspiration inlet, the aspiration conduit comprising a body with distal end engaged to the acquisition device and a proximal end comprising a first connector;
a sample tube comprising a body with a distal end and a proximal end, the distal end comprising a second connector that connects to the first connector; and
an aspirator engaged to the proximal end of the sampling tube, the aspirator comprising a valve for shutting off aspiration; wherein the aspiration tube and the sampling tube are configured to handle a minimum sample volume.

37. The system of claim 36, wherein the sampling tube comprises at least one indicator positioned at a predetermined location on the body of the sampling tube.

38. The system of claim 37, wherein the at least one indicator comprises two indicators each positioned on the body of the sampling tube for indicating a desired sample volume.

39. (canceled)

40. The system of claim 36, further comprising an analyte detection device comprising a housing with a proximal end and distal end, the proximal end comprising a port, wherein upon disconnecting the first and second connectors, the second connector connects to the port of the analyte detection device.

41. The system of claim 40, wherein the analyte detection device comprises a sample staging chamber in fluid communication with the port and an overflow passage in fluid communication with the sample staging chamber.

42. The system of claim 41, wherein the analyte detection device further comprises a sample transfer chamber, a test chamber and a sample pad that is disposed in the sample staging chamber, sample transfer chamber and test chamber for transferring a sample from the sample staging chamber to the test chamber; a conjugate pad disposed in the sample transfer chamber and test chamber that contacts the sample pad, and an assay platform disposed in the test chamber that contacts the conjugate pad, the assay platform comprising a test region and a control region.

43. (canceled)

44. (canceled)

45. The system of claim 42, further comprising a wicking chamber and a wicking pad disposed within the wicking chamber and test chamber, wherein the wicking pad contacts the assay platform.

46. The system of claim 44, further comprising a vent in fluid communication with the overflow passage.

47. (canceled)

48. A method for detecting at least one analyte in a vitreous sample comprising obtaining a system of claim 40;
aspirating the vitreous humor or aqueous humor sample by applying vacuum to the sampling tube while the first and second connectors are connected and the sample acquisition device is in an eye;
when the vitreous humor or aqueous humor sample reaches the at least one indicator, closing the valve;
disconnecting the first and second connectors and engaging the second connector to the port; and
applying pressure to the sampling tube to deliver the vitreous sample to the analyte detection device.

49. (canceled)

50. A system for detecting an analyte in a vitreous humor or aqueous humor sample, the system comprising
a sampling tube comprising a body with a distal end and a proximal end,
an acquisition device comprising a cannulated needle with an aspiration inlet, the aspiration inlet in fluid communication with the distal end of the sampling tube;
an aspirator engaged to the proximal end of the sampling tube;
a sample conduit in fluid communication with the body of the sampling tube; and
an analyte detection device in fluid communication with the sample conduit;
wherein the sampling tube comprises a first valve upstream to the sample conduit and the sample conduit comprises a second valve such that during the aspiration of a sample the first valve opens and the second valve closes, and during transfer of aspirated sample to the analyte detection device, the second valve opens and the first valve closes.
